SEQUENCE LISTING (15 sequences)

SEQ ID NO: 1 - DNA, 2322 bp, Homo sapiens
gggagggggtgggatgggtggggggtaacgggggaaactggggaagtggggaaccgaggggcaaccaggggaagatggggtgctggaggagagcttgtgggagccaaggagcaccttggacatctggagtctggcaggagtgatgacgggtggaggggctagctcgaggcagggctggtggggcctgaggccagtgaggagtgtggagtaggcgcccaggcatcgtgcagacagggcgacatcagctggggacgatgggcctgagctagggctggaaagaagggggagccaggcattcatcccggtcacttttggttacaggacgtggcagctggttggacgaggggagctggtgggcagggtttgatcccagggcctgggcaacggaggtgtagctggcagcagcgggcaggtgaggaccccatctgccgggcaggtgagtcccttccctccccaggcctcgcttccccagccttctgaaagaaggaggtttaggggatcgagggctggcggggagaagcagacaccctcccagcagaggggcaggatgggggcaggagagttagcaaaggtgacatcttctcggggggagccgagactgcgcaaggctggggggttatgggcccgttccaggcagaaagagcaagagggcagggagggagcacaggggtggccagcgtagggtccagcacgtggggtggtaccccaggcctgggtcagacagggacatggcaggggacacaggacagaggggtccccagctgccacctcacccaccgcaattcatttagtagcaggcacaggggcagctccggcacggctttctcaggcctatgccggagcctcgagggctggagagcgggaagacaggcagtgctcggggagttgcagcaggacgtcaccaggagggcgaagcggccacgggaggggggccccgggacattgcgcagcaaggaggctgcaggggctcggcctgcgggcgccggtcccacgaggcactgcggcccagggtctggtgcggagagggcccacagtggacttggtgacgctgtatgccctcaccgctcagcccctggggctggcttggcagacagtacagcatccaggggagtcaagggcatggggcgagaccagactaggcgaggcgggcggggcggagtgaatgagctctcaggagggaggatggtgcaggcaggggtgaggagcgcagcgggcggcgagcgggaggcactggcctccagagcccgtggccaaggcgggcctcgcgggcggcgacggagccgggatcggtgcctcagcgttcgggctggagacgaggccaggtctccagctggggtggacgtgcccaccagctgccgaaggccaagacgccaggtccggtggacgtgacaagcaggacatgacatggtccggtgtgacggcgaggacagaggaggcgcgtccggccttcctgaacaccttaggctggtggggctgcggcaagaagcgggtctgtttctttacttcctccacggagtcggcacactatggctgccctctgggctcccagaacccacaacatgaaagaaatggtgctacccagctcaagcctgggcctttgaatccggacacaaaaccctctagcttggaaatgaatatgctgcactttacaaccactgcactacctgactcaggaatcggctctggaaggtgaagctagaggaaccagacctcatcagcccaacatcaaagacaccatcggaacagcagcgcccgcagcacccaccccgcaccggcgactccatcttcatggccaccccctgcggcggacggttgaccaccagccaccacatcatcccagagctgagctcctccagcgggatgacgccgtccccaccacctccctcttcttctttttcatccttctgtctctttgtttctgagctttcctgtctttccttttttctgagagattcaaagcctccacgactctgtttcccccgtcccttctgaatttaatttgcactaagtcatttgcactggttggagttgtggagacggccttgagtctcagtacgagtgtgcgtgagtgtgagccaccttggcaagtgcctgtgcagggcccggccgccctccatctgggccgggtgactgggcgccggctgtgtgcccgaggcctcaccctgccctcgcctagtctggaagctccgaccgacatcacggagcagccttcaagcattccattacgccccatctcgctctgtgcccctccccaccagggcttcagcaggagccctggactcatcatcaataaacactgttacagcaaaaaaaaaaaaaaaa

SEQ ID NO: 2 - DNA, 22 bp, Artificial Sequence
  Single strand DNA oligonucleotide
ccgactcgagnnnnnnatgtgg

SEQ ID NO: 3 - DNA, 6129 bp, Homo sapiens
  misc_feature (1496)..(1496): can be C or T
aattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggnggttgctggatccactggagcaggcaagacttcacttctaatgatgattatgggagaactggagccttcagagggtaaaattaagcacagtggaagaatttcattctgttctnagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatattacaatgctgtattttaaaagaatgattatgaattacatttgtataaaataatttttatatttgaaatattgactttttatggcactagtatttttatgaaatattatgttaaaactgggacaggggagaacctagggtgatattaaccaggggccatgaatcaccttttggtctggagggaagccttggggctgatcgagttgttgcccacagctgtatgattcccagccagacacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgt

SEQ ID NO: 4 - DNA, 22 bp, Artificial Sequence
  Single strand DNA oligonucleotide
gcaccattaaagaaaatatgat

SEQ ID NO: 5 - DNA, 19 bp, Artificial Sequence
  Single strand DNA oligonuclietide
ctcttctagttggcatgct

SEQ ID NO: 6 - DNA, 20 bp, Artificial Sequence
  Single strand DNA oligonucleotide
taatggatcatgggccatgt

SEQ ID NO: 7 - DNA, 20 bp, Artificial Sequence
  Single strand DNA oligonucleotide
acagtgttgaatgtggtgca

SEQ ID NO: 8 - DNA, 16 bp, Artificial Sequence
  Single strand DNA oligonucleotide
gttgttggaggttgct

SEQ ID NO: 9 - DNA, 16 bp, Artificial Sequence
  Single strand DNA oligonucleotide
gttgttggcggttgct

SEQ ID NO: 10 - DNA, 20 bp, Artificial Sequence
  Single strand DNA oligonucleotide
gcagagtacctgaaacagga

SEQ ID NO: 11 - DNA, 20 bp, Artificial Sequence
  Single strand DNA oligonucleotide
ggcataatccaggaaaactg

SEQ ID NO: 12 - DNA, 20 bp, Artificial Sequence
  Single strand DNA oligonucleotide
ggcataatccaggaaaacta

SEQ ID NO: 13 - RNA, 50 nt, Artificial Sequence
  Synthetic RNA oligonecleotide
cguaauggaaugcuugaaggcugcuccgugaugucggucggagcuuccag

SEQ ID NO: 14 - DNA, 28 bp, Artificial Sequence
  Single strand DNA oligonucleotide
gaaactggcctccaaacactgcccgccg

SEQ ID NO: 15 - DNA, 29 bp, Artificial Sequence
  Single strand DNA oligonucleotide
gtcttgttggagatgcacgtgccccttgc

(19) United States
(12) Patent Application Publication
(10) Pub. No.: US 2010/0035246 A1
(43) Pub. Date: Feb. 11, 2010

(54) Methods and Kits for Analyzing Genetic Material of a Fetus

(76) Inventors: Avner Lushi, Ramat Gan (IL); Hadar Ron, Tel Aviv (IL); Boris Tartakovsky, Rehovot (IL); Moshe Fejgin, Tel Aviv (IL); Aliza Amiel, Moshav Ein Sarid (IL)

Correspondence Address: THE NATH LAW GROUP, 112 South West Street, Alexandria, VA, 22314, US

(21) Appl. No.: 12/083,868

(22) PCT Filed: Oct. 22, 2006
(86) PCT No.: PCT/IL2006/001212

§ 371 Date: Jun. 30, 2009

Related U.S. Application Data
(60) Provisional application No. 60/728,799, filed on Oct. 21, 2005.

(51) Int. Cl. C12Q 1/68; C12Q 1/02

(52) U.S. Cl. 435/6; 435/29

(57) ABSTRACT

A non-invasive method of analyzing a genetic material of a fetus is provided. The method is achieved by detecting a cell-free nucleus in a sample such as a transcervical specimen obtained from a pregnant woman and/or detecting in a cell-free nucleus at least one fetal-nucleus specific marker thereby identifying a fetal nucleus; and molecularly analyzing the genetic material in the fetal nucleus, thereby analyzing the genetic material of the fetus.

### FIELD OF THE INVENTION

The present invention relates to the identification of cell-free fetal nuclei in samples obtained from pregnant women, and more particularly, to a non-invasive method of prenatal diagnosis using same.

### BACKGROUND OF INVENTION

Prenatal diagnosis involves the identification of major or minor fetal malformations or genetic diseases present in a human fetus. Ultrasound scans can usually detect structural malformations such as those involving the neural tube, heart, kidney, limbs and the like. On the other hand, chromosomal aberrations such as presence of extra chromosomes [e.g., Trisomy 21 (Down syndrome); Klinefelter's syndrome (47, XXY); Trisomy 13 (Patau syndrome); Trisomy 18 (Edwards syndrome); 47, XYY; 47, XXX], the absence of chromosomes [e.g., Turner's syndrome (45, X0)], or various translocations and deletions can be currently detected using chorionic villus sampling (CVS) and/or amniocentesis.

Currently, prenatal diagnosis is offered to women over the age of 35 and/or to women which are known carriers of genetic diseases such as balanced translocations or microdeletions (e.g., Di-George syndrome), and the like. Thus, the percentage of women over the age of 35 who give birth to babies with chromosomal aberrations to such as Down syndrome has drastically reduced. However, the lack of prenatal testing in younger women resulted in the surprising statistics that 80% of Down syndrome babies are actually born to women under the age of 35.

CVS is usually performed between the 9^{th} and the 14^{th} week of gestation by inserting a catheter through the cervix or a needle into the abdomen and removing a small sample of the placenta (i.e., chorionic villus). Fetal karyotype is usually determined within one to two weeks of the CVS procedure. However, since CVS is an invasive procedure it carries a 2-4% procedure-related risk of miscarriage and may be associated with an increased risk of fetal abnormality such as defective limb development, presumably due to hemorrhage or embolism from the aspirated placental tissues (Miller D, et al, 1999. Human Reproduction 2: 521-531)

On the other hand, amniocentesis is performed between the 16^{th} to the 20^{th} week of gestation by inserting a thin needle through the abdomen into the uterus. The amniocentesis procedure carries a 0.5-1.0% procedure-related risk of miscarriage. Following aspiration of amniotic fluid, the fetal fibroblast cells are further cultured for 1-2 weeks, following which they are subjected to cytogenetic (e.g., G-banding) and/or FISH analyses. Thus, fetal karyotype analysis is obtained within 2-3 weeks of sampling the cells. However, in cases of abnormal findings, the termination of pregnancy usually occurs between the 18^{th} to the 22^{nd} week of gestation, involving the Boero technique, a more complicated procedure in terms of psychological and clinical aspects.

The discovery of fetal nucleated erythrocytes in the maternal blood early in gestation has prompted many investigators to develop methods of isolating these cells and subjecting them to genetic analysis (e.g., PCR, FISH). However, since the frequency of nucleated fetal cells in the maternal blood is exceptionally low (0.0035%), the NRBC cells had to be first purified (e.g., using Ficol-Paque or Percoll-gradient density centrifugation) and then enriched using for example, magnetic activated cell sorting (MACS, Busch, J. et al., 1994, Prenat. Diagn. 14: 1129-1140), ferrofluid suspension (Steele, C. D. et al., 1996, Clin. Obstet. Gynecol. 39: 801-813), charge flow separation (Wachtel, S. S. et al., 1996, Hum. Genet. 98:162-166), or FACS analysis (Wang, J. Y. et al., 2000, Cytometry 39:224-230).

U.S. Pat. No. 5,750,339 discloses genetic analysis of fetal NRBCs cells derived from the maternal. According to the approach disclosed therein, the fetal cells are enriched using anti CD71, CD36 and/or glycophorin A and the maternal cells are depleted using anti-maternal antibodies such as anti-CD14, CD4, CD8, CD3, CD19, CD32, CD16 and CD4. Resultant fetal cells are identified using an HLA-G specific probe. Although recovery of fetal NRBCs can be achieved using such an approach, inconsistent recovery rates coupled with limited sensitivity prevents clinical application of diagnostic techniques using fetal NRBCs (Bischoff, F. Z. et al., 2002. Hum. Repr. Update 8: 493-500).

Various studies attempted to isolate fetal trophoblasts from the maternal blood (WO 9915892A1 Pat. Appl. to Kalionis B; U.S. Pat. Appl. No. 20050049793 to Paterlini-Brechot, P., et al.; US Pat. Appl. Nos. 20020045196A1, 20030013123 and EP Pat. Appl. No. 1154016A2 to Mahoney W. et al.). However, all of these studies resulted in inconsistent results due to the isolation of mixed cells in which the genetic origin (i.e., maternal or fetal) was uncertain.

Other studies describe the identification of trophoblast cells in transcervical specimens using a variety of antibodies such as HLA-G (Bulmer, J. N. et al., 2003. Prenat. Diagn. 23: 34-39), PLAP, FT1.41.1, NDOG-1, NDOG-5, and 340 (Miller et al., 1999. Human Reproduction, 14: 521-531). In these studies the antibodies truly recognized trophoblast cells in 30-79% of the transcervical specimens. In addition, the FISH, PCR and/or quantitative fluorescent PCR (QF-PCR) analyses, which were performed on duplicated transcervical specimens, were capable of identifying approximately 80-90% of all male fetuses. However, since the DNA (e.g., FISH and/or PCR) and immunological (e.g., immunohistochemical, IHC) analyses were performed on separated slides, these methods were impractical for diagnosing fetal chromosomal abnormalities. Other studies utilizing magnetic activating cell sorting on transcervical trophoblasts resulted in mixed populations of fetal and maternal cells, limiting their use in prenatal diagnosis (PCT WO04076653A1 to Irwin DL., et al.).

The present inventors have previously disclosed non-invasive methods for prenatal diagnosis using trophoblast cells present in transcervical specimens (U.S. Pat. Publication Nos. 20040197832, 20050003351, 20050181429 and PCT Publication No. WO04087863A2). According to these methods, the trophoblast cells are first identified by immunological or RNA-in situ hybridization staining methods using antibodies or probes specific to fetal cells and then are subjected to a molecular analysis (using e.g., FISH, DNA-based analysis) which allows, with high accuracy, the identification of chromosomal and/or DNA abnormalities in the fetus.

Recently, cell-free fetal DNA circulating in the maternal plasma was used for determining fetal gender or Rhesus (Rh)-D (RhD) status (Brojer E., et al., 2005, Transfusion, 45: 1473-80; Costa J M., et al., 2004, Gynecol. Obstet. Fertil. 32: 646-50) or point mutations for beta-thalassemia (Li Y, et al., 2005, JAMA, 293: 843-9).

However, to date, the use of cell-free fetal nuclei present in maternal samples for prenatal diagnosis has never been suggested or shown.

### SUMMARY OF INVENTION

According to one aspect of the present invention there is provided a method of identifying a fetal nucleus, comprising: (a) detecting a cell-free nucleus in a sample; and/or (b) detecting in a cell-free nucleus at least one fetal-nucleus specific marker; thereby identifying the fetal nucleus.

According to another aspect of the present invention there is provided a method of analyzing a genetic material of a fetus, comprising: (a) detecting a cell-free nucleus in a sample and/or; (b) detecting in a cell-free nucleus at least one fetal-nucleus specific marker; thereby identifying a fetal nucleus; and (c) molecularly analyzing the genetic material in the fetal nucleus; thereby analyzing the genetic material of the fetus.

According to yet another aspect of the present invention there is provided a kit for analyzing a genetic material of a fetus, comprising a packaging material packaging a reagent for detecting at least one fetal-nucleus specific marker.

According to further features in preferred embodiments of the invention described below, the sample is a trophoblast-containing sample obtained from a pregnant woman.

According to still further features in the described preferred embodiments the trophoblast-containing sample is obtained from a cervix and/or a uterus of the pregnant woman.

According to still further features in the described preferred embodiments the trophoblast-containing sample is obtained using a method selected from the group consisting of aspiration, cytobrush, cotton wool swab, endocervical lavage and intrauterine lavage.

According to still further features in the described preferred embodiments the sample is a blood sample obtained from a pregnant woman.

According to still further features in the described preferred embodiments the at least one fetal-nucleus specific marker is a molecular marker.

According to still further features in the described preferred embodiments the molecular marker is selected from the group consisting of a nucleic acid marker and a protein marker.

According to still further features in the described preferred embodiments the nucleic acid marker is a nuclear RNA molecule.

According to still further features in the described preferred embodiments the nuclear RNA molecule is an H19 transcript.

According to still further features in the described preferred embodiments the nucleic acid marker is an epigenetic marker.

According to still further features in the described preferred embodiments the epigenetic marker is located on H19 and/or IGF2.

According to still further features in the described preferred embodiments the nuclear RNA molecule is an hnRNA transcript encoding a polypeptide selected from the group consisting of ESX1L, MASH2, Ash2, Stra 13, FosB, Cyclin D1, GCM1 and Caspase-8.

According to still further features in the described preferred embodiments the protein marker is a trophoblast specific antigen selected from the group consisting of ESX1L, MASH2, Ash2, Stra 13, FosB, Cyclin D1, GCM1 and Caspase-8.

According to still further features in the described preferred embodiments the detection of the nuclear RNA molecule comprises using an RNA in situ hybridization (RNA-ISH) staining.

According to still further features in the described preferred embodiments the RNA-ISH staining comprises using a probe selected from the group consisting of an RNA molecule, a DNA molecule and a PNA oligonucleotide.

According to still further features in the described preferred embodiments the RNA molecule is an RNA oligonucleotide and/or an in vitro transcribed RNA.

According to still further features in the described preferred embodiments the DNA molecule is an oligonucleotide and/or a cDNA molecule.

According to still further features in the described preferred embodiments detecting the protein marker comprises using an immunological staining.

According to still further features in the described preferred embodiments the trophoblast-containing sample is obtained from a pregnant woman at 5th to 15th week of gestation.

According to still further features in the described preferred embodiments identifying the at least one cell-free nucleus in the sample is achieved by image analysis.

According to still further features in the described preferred embodiments the molecularly analyzing the genetic material comprises using an approach selected from the group consisting of an in situ chromosomal analysis, an in situ DNA analysis and a genetic analysis.

According to still further features in the described preferred embodiments the in situ chromosomal analysis comprises using fluorescent in situ hybridization (FISH) and/or multicolor-banding (MCB).

According to still further features in the described preferred embodiments in situ DNA analysis comprises using primed in situ labeling (PRINS) and/or quantitative FISH (Q-FISH).

According to still further features in the described preferred embodiments the Q-FISH comprises using a peptide nucleic acid (PNA) oligonucleotide probe.

According to still further features in the described preferred embodiments the genetic analysis utilizes at least one method selected from the group consisting of comparative genome hybridization (CGH) and identification of at least one nucleic acid substitution.

According to still further features in the described preferred embodiments, the method further comprising a step of isolating the at least one fetal nucleus prior to step (c).

According to still further features in the described preferred embodiments the isolating the at least one fetal nucleus is achieved using laser microdissection.

According to still further features in the described preferred embodiments the identification of at least one nucleic acid substitution is achieved using a method selected from the group consisting of DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, Invader assay, MLPA and MS-MLPA.

According to still further features in the described preferred embodiments the identifying the at least one cell-free nucleus in the sample is achieved by image analysis.

According to still further features in the described preferred embodiments analysis of the genetic material of the fetus enables the identification of fetal gender, at least one chromosomal abnormality, at least one DNA abnormality and/or a paternity of the fetus.

According to still further features in the described preferred embodiments the at least one chromosomal abnormality is selected from the group consisting of aneuploidy, translocation, subtelomeric rearrangement, unbalanced subtelomeric rearrangement, deletion, microdeletion, inversion, duplication, and telomere instability and/or shortening.

According to still further features in the described preferred embodiments the chromosomal aneuploidy is a complete and/or partial trisomy.

According to still further features in the described preferred embodiments the trisomy is selected from the group consisting of trisomy 21, trisomy 18, trisomy 13, trisomy 16, XXY, XYY, and XXX.

According to still further features in the described preferred embodiments the chromosomal aneuploidy is a complete and/or partial monosomy.

According to still further features in the described preferred embodiments the monosomy is selected from the group consisting of monosomy X, monosomy 21, monosomy 22, monosomy 16 and monosomy 15.

According to still further features in the described preferred embodiments the at least one DNA abnormality is selected from the group consisting of single nucleotide substitution, micro-deletion, micro-insertion, short deletions, short insertions, multinucleotide changes, DNA methylation and loss of imprint (LOI).

According to still further features in the described preferred embodiments the genetic material of the fetus is derived from a cell-free fetal nucleus.

According to still further features in the described preferred embodiments detection of the nuclear RNA molecule comprises using an RNA in situ hybridization (RNA-ISH) staining.

According to still further features in the described preferred embodiments the kit further comprising a second reagent suitable for a molecular analysis of the genetic material of the fetus, the molecular analysis is selected from the group consisting of an in situ chromosomal analysis, an in situ DNA analysis and a genetic analysis.

The present invention successfully addresses the shortcomings of the presently known configurations by providing a non-invasive prenatal diagnosis method using fetal cell nuclei present in a transcervical specimen.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### DETAILED DESCRIPTION OF THE DRAWINGS

In order to understand the invention and to see how it may be carried out in practice, a preferred embodiment will now be described, by way of non-limiting example only, with reference to the accompanying drawings, in which:

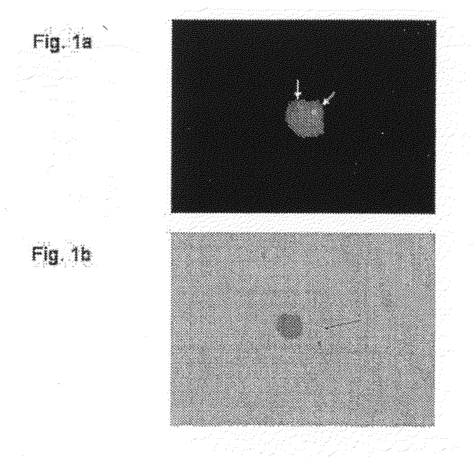

FIGS. 1a-b are photomicrographs illustrating FISH analysis (FIG. 1a) and Hematoxylin counterstaining (FIG. 1b) of cell-free nuclei from transcervical specimens. Shown is a cell-free nucleus stained with Hematoxylin (FIG. 1b, arrow) and following FISH analysis (FIG. 1a). Note the single orange (left) and green (right) signals in the cell-free nucleus (FIG. 1a, arrows) corresponding to the Y and X chromosomes, respectively, demonstrating the identification of a normal male fetus using a cell-free fetal nucleus present in a transcervical specimen.

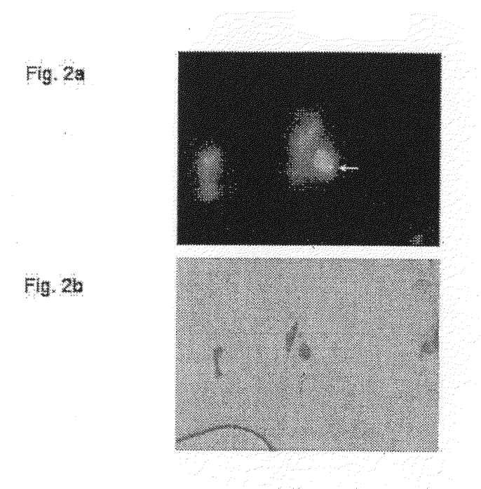

FIGS. 2a-b are photomicrographs illustrating FISH analysis (FIG. 2a) and Hematoxylin counterstaining (FIG. 2b) of cell-free nuclei from transcervical specimens. Shown is a cell-free nucleus stained with Hematoxylin (FIG. 2b, arrow) and following FISH analysis (FIG. 2a). Note the single orange and green signals in the cell-free nucleus (FIG. 2a, arrow) corresponding to the Y and X chromosomes, respectively, demonstrating the identification of a normal male fetus using a cell-free fetal nucleus present in a transcervical specimen.

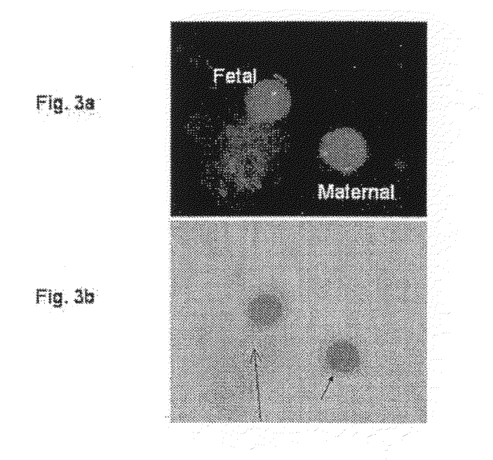

FIGS. 3a-b are photomicrographs illustrating FISH analysis (FIG. 3a) and Hematoxylin counterstaining (FIG. 3b) of cell-free nuclei from transcervical specimens. Shown are cell-free nuclei stained with Hematoxylin (FIG. 3b, arrows) and following FISH analysis (FIG. 3a). Note the presence of one fetal cell-free nucleus (FIG. 3a, nucleus marked with “Fetal”) with both orange and green signals corresponding to the Y and X chromosomes, respectively, and one maternal cell-free nucleus (FIG. 3a, nucleus marked with “Maternal”) with two green signals corresponding to two X chromosomes.

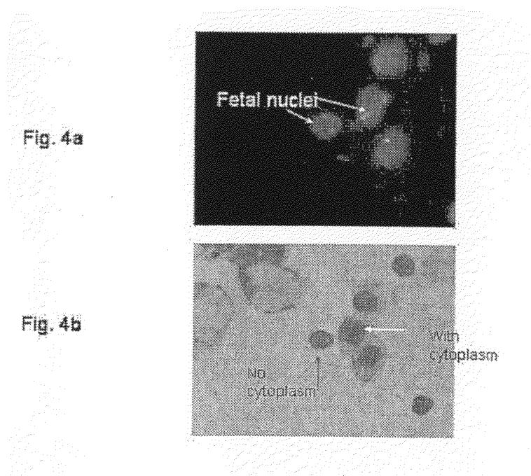

FIGS. 4a-b are photomicrographs illustrating FISH analysis (FIG. 4a) and Hematoxylin counterstaining (FIG. 4b) of cells and cell-free nuclei from transcervical specimens. Shown are one cell-free nucleus (FIG. 6b, dark arrow) and three whole cells (FIG. 6b, white arrow) stained with Hematoxylin and following FISH analysis (FIG. 6a). Note the presence of both orange and green signals in the cell-free nuclei (FIG. 6a, nuclei marked “Fetal nuclei”) corresponding to the Y and X chromosomes, respectively, demonstrating the identification of a male fetus from the fetal cell-free nucleus.

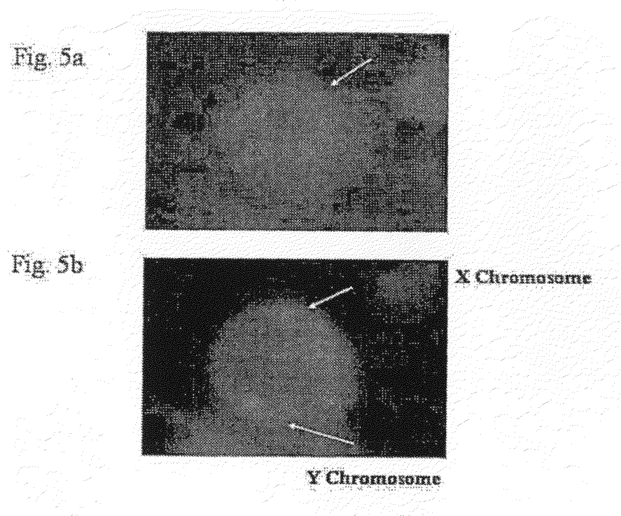

FIGS. 5a-b are photomicrographs illustrating Ash2 protein nuclear membrane staining (FIG. 5a) and FISH analysis (FIG. 5b) of a cell-free nucleus from transcervical specimens. Transcervical samples were subjected to immunofluorescence using an anti Ash 2 antibody (rabbit polyclonal, Novus NB600-253) followed by FISH analysis using the CEP X spectrum green and CEP Y spectrum orange (Abbott, Cat. 5J10-51) probes and DAPI counterstaining. Shown is one cell-free nucleus stained with the Ash2 antibody (FIG. 5a) and following FISH analysis (FIG. 5b). Note the presence of both orange and green signals in the cell-free nucleus (FIG. 5b) corresponding to the Y and X chromosomes, respectively, demonstrating the identification of a male fetus based on the fetal cell-free nucleus staining.

### DETAILED DESCRIPTION OF EXEMPLARY EMBODIMENTS OF THE INVENTION

The present invention relates to the identification of cell-free fetal nuclei in transcervical specimens which can be used for prenatal diagnosis. Specifically, the present invention uses an agent capable of identifying a fetal nucleus specific marker such as H19 for the identification of cell-free fetal nuclei which are further used for molecularly analyzing the genetic material of the fetus.

The principles and operation of the method of analyzing the genetic material of a fetus according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Prenatal diagnosis involves the identification of major or minor fetal malformations or genetic diseases present in a human fetus. Chromosomal aberrations such as presence of extra chromosomes [e.g., Trisomy 21 (Down syndrome); Klinefelter's syndrome (47, XXY); Trisomy 13 (Patau syndrome); Trisomy 18 (Edwards syndrome); 47, XYY; 47, XXX], the absence of chromosomes [e.g., Turner's syndrome (45, X0)], or various translocations and deletions are currently detected using chorionic villus sampling (CVS) or amniocentesis. However, although desired, such invasive procedures carry a procedure-related risk of miscarriage of 2-4% or 0.5-1.0%, respectively.

Prior studies have uncovered the presence of fetal cells in maternal-derived samples. These include fetal nucleated erythrocytes (NRBCs; U.S. Pat. No. 5,750,339; Bischoff, F. Z. et al., 2002. Hum. Repr. Update 8: 493-500), trophoblast cells in the maternal blood (WO 9915892A1 Pat. Appl. to Kalionis B; U.S. Pat. Appl. No. 20050049793 to Paterlini-Brechot, P., et al.; US Pat. Appl. Nos. 20020045196A1, 20030013123 and EP Pat. Appl. No. 1154016A2 to Mahoney W. et al.) and trophoblast cells in transcervical specimens (Miller et al., 1999. Human Reproduction, 14: 521-531).

The present inventors have previously disclosed non-invasive methods for prenatal diagnosis using trophoblast cells present in transcervical specimens (U.S. Pat. Publication. Nos. 20040197832, 20050003351, 20050181429 and PCT Publication No. WO04087863A2). According to these methods, the trophoblast cells are first identified by immunological or RNA-in situ hybridization staining methods using antibodies or probes specific to fetal cells and are then subjected to a molecular analysis (using e.g., FISH, DNA-based analysis) which allows, with relatively high accuracy, the identification of chromosomal and/or DNA abnormalities in the fetus. However, these methods are limited by the availability of trophoblast cells in transcervical samples.

Recently, cell-free fetal DNA circulating in the maternal plasma was used for determining fetal RhD status (Brojer E., et al., 2005, Transfusion, 45: 1473-80) or point mutations for beta-thalassemia (Li Y, et al., 2005, JAMA, 293: 843-9).

While reducing the present invention to practice, the present inventors have uncovered that multiple cell-free fetal nuclei are present in transcervical specimens obtained from pregnant women and that the relative representation of the fetal cell-free nuclei in the transcervical specimen is much higher than that of the fetal cells.

Thus, according to one aspect of the present invention, there is provided a method of identifying a fetal nucleus. The method is achieved by (a) detecting a cell-free nucleus in a sample; and/or (b) detecting in a cell-free nucleus at least one fetal-nucleus specific marker; thereby identifying the fetal nucleus.

The phrase “fetal nucleus” refers to a nucleus of a cell which is derived from the fetus. Non-limiting examples of such cells include trophoblasts, fetal nucleated red blood cells and fetal leukocyte cells.

The phrase “cell-free nucleus” refers to a nucleus which has lost its surrounding cytoplasm, such as due to membrane rupture, apoptosis, enhanced osmosis and the like. The phrase “detecting” as used herein refers to identifying (e.g., visually) and/or isolating (e.g. physically) the cell-free nucleus of the present invention. It will be appreciated that since the representation of the fetal cell-free nuclei in transcervical specimens is relatively high (as compared with that of fetal cells) detection of such a cell-free nucleus can be easily performed by one skilled in the art using a microscope (e.g., a light microscope), with or without an image analysis apparatus (e.g., the BioView Duet™, Rehovot, Israel) and/or a CCD camera. Alternatively or additionally, physical isolation of cell-free nuclei can be also performed such as by using specific antibodies, gradient separation (e.g., by density), filters, and/or microdissection (e.g., laser capture microdissection) as is further described herein below.

The sample used by the method according to this aspect of the present invention is obtained from a pregnant woman at any stage of the pregnancy. Such a sample is preferably a trophoblast-containing sample which includes a cell-free trophoblast nucleus.

The term “trophoblast” refers to an epithelial cell which is derived from the placenta of a mammalian embryo or fetus; trophoblasts typically contact the uterine wall. There are three types of trophoblast cells in the placental tissue: the villous cytotrophoblast, the syncytiotrophoblast, and the extravillous trophoblast, and as such, the term “trophoblast” as used herein encompasses any of these cells. The villous cytotrophoblast cells are specialized placental epithelial cells which differentiate, proliferate and invade the uterine wall to form the villi. Cytotrophoblasts, which are present in anchoring villi can fuse to form the syncytiotrophoblast layer or form columns of extravillous trophoblasts (Cohen S. et al., 2003. J. Pathol. 200: 47-52).

The cell-free trophoblast nucleus containing samples can be obtained from any biological sample derived from a pregnant woman at various stages of gestation including a blood sample, a transcervical and/or intrauterine sample, an amniotic fluid sample and/or a CVS sample. Preferably, the cell-free trophoblast nucleus is obtained using a non-invasive method, e.g., by drawing maternal blood or obtaining a specimen from the cervix and/or the uterus of a pregnant woman (i.e., a transcervical and/or an intrauterine specimen, respectively).

The cell-free trophoblast nucleus-containing sample utilized by the method of the present invention can be obtained using any one of numerous well known cell collection techniques.

According to preferred embodiments of the present invention the cell-free trophoblast nucleus-containing sample is obtained using mucus aspiration (Sherlock, J., et al., 1997. J. Med. Genet. 34: 302-305; Miller, D. and Briggs, J. 1996. Early Human Development 47: S99-S102), cytobrush (Cioni, R., et al., 2003. Prent. Diagn. 23: 168-171; Fejgin, M. D., et al., 2001. Prenat. Diagn. 21: 619-621), cotton wool swab (Griffith-Jones, M. D., et al., 1992 Br J Obstet. Gynaecol. 99 (6): 508-511), endocervical lavage (Massari, A., et al., 1996. Hum. Genet. 97: 150-155; Griffith-Jones, M. D., et al., 1992. Supra; Schueler, P. A. et al., 2001. 22: 688-701), and intrauterine lavage (Cioni, R., et al., 2002. Prent. Diagn. 22: 52-55; Ishai, D., et al., 1995. Prenat. Diagn. 15: 961-965; Chang, S-D., et al., 1997. Prenat. Diagn. 17: 1019-1025; Sherlock, J., et al., 1997, Supra; Bussani, C., et al., 2002. Prenat. Diagn. 22: 1098-1101). See for comparison of the various approaches Adinolfi, M. and Sherlock, J. (Human Reprod. Update 1997, 3: 383-392 and J. Hum. Genet. 2001, 46: 99-104), Rodeck, C., et al. (Prenat. Diagn. 1995, 15: 933-942). The cytobrush method is the presently preferred method of obtaining the trophoblast cell-free nucleus containing sample of the present invention.

In the cytobrush method, a Pap smear cytobrush (e.g., MedScand-AB, Malm6, Sweden) is inserted through the external os to a maximum depth of 2 cm and removed while rotating it a full turn (i.e., 360° ). In order to remove the transcervical cell-free nuclei caught on the brush, the brush is shaken into a test tube containing 2-3 ml of a tissue culture medium (e.g., RPMI-1640 medium, available from Beth Haemek, Israel) in the presence of 1% Penicillin Streptomycin antibiotic. In order to concentrate the transcervical cell-free nuclei on microscopic slides cytospin slides are prepared using e.g., a Cytofunnel Chamber Cytocentrifuge (Thermo-Shandon, England). It will be appreciated that the conditions used for cytocentrifugation are dependent on the murkiness of the transcervical specimen; if the specimen contained only a few cell-free nuclei, the cell-free nuclei are first centrifuged for 5 minutes and then suspended with 1 ml of fresh medium. Once prepared, the cytospin slides can be kept in 95% alcohol until further use.

Since cell-free trophoblast nuclei are shed from the placenta into the uterine cavity, the nucleus containing sample of the present invention should be retrieved as long as the uterine cavity persists, which is until about the 13-15 weeks of gestation (reviewed in Adinolfi, M. and Sherlock, J. 2001, Supra).

Thus, according to preferred embodiments of the present invention the cell-free trophoblast nucleus-containing sample is obtained from a pregnant woman at 3^{rd} to 15^{th} week of gestation. Preferably, the cell-free trophoblast nucleus-containing sample is obtained from a pregnant woman between the 4^{th} to 15^{th} week of gestation, more preferably, between the 5^{th} to 15^{th} week of gestation, more preferably, between the 6^{th} to the 13^{th} week of gestation, more preferably, between the 6^{th} to the 11^{th} week of gestation, even more preferably between the 6^{th} to the 10^{th} week of gestation.

It will be appreciated that the determination of the exact week of gestation during a pregnancy is well within the capabilities of one of ordinary skill in the art of Gynecology and Obstetrics.

According to this aspect of the present invention, once detected, the cell-free nucleus is subject to the detection of at least one fetal-nucleus specific marker which enables the identification of its genetic background (i.e., maternal or fetal).

As used herein the phrase “fetal-nucleus specific marker” refers to any molecule such as a nucleic acid (i.e., DNA, RNA and modifications or derivatives thereof), a protein, a carbohydrate, a lipid or combination thereof (e.g., a glycosylated protein, a proteoglycan) which is specifically present in the nucleus of fetal cells but is missing from the nucleus of maternal cells. Preferably, such a marker is a molecular marker such as a nucleic acid marker and/or a protein marker.

The nucleic acid marker of the present invention can be for example, a nuclear RNA marker [i.e., an RNA molecule which is present in the nucleus and/or nucleoli of a fetal cell, such as H19 (Lin W L, et al., 1999, Mech. Dev. 82: 195-7); SEQ ID NO: 1; GenBank Accession No. NR_{—}002196], an epigenetic marker capable of identifying physical or chemical changes in the chromatin of fetal cells as compared with the chromatin of maternal cells such as differential methylation of specific CpG islands in imprinted genes (e.g., H19 and IGF2; Poon L L., et al., 2002, Clin. Chem. 48: 35-41), and/or a heteronuclear RNA (hnRNA) transcript (i.e., the primary transcript of an mRNA molecule before splicing thereof) which encodes a fetal-specific polypeptide.

Fetal specific nuclear markers (e.g., polypeptides) can be identified by experimental and/or data mining methods. For example, placental tissue or trophoblast-derived cell lines (Shi F, et al., 1997, Exp. Cell Res. 234: 147-55; Rong-Hao L, et al., 1996, Hum. Reprod. 11: 1328-33; Ho C K, et al., 1987, Cancer Res. 47: 3220-4) can be subjected to molecular methods such as RT-PCR analysis (as described in Lowndes K, et al., Placenta. 2005 Sep. 12; [Epub ahead of print]; Chen Y T, et al., 2005, Cancer Immun. 5:9), RNA-ISH (as is further described hereinbelow), Western blot analysis (Kam D W, et al., 2005, Reprod Biomed Online. 11: 236-43), immunohistochemistry and/or immunofluorescence (as described hereinbelow and in Briese J, et al., 2005, J. Clin. Endocrinol. Metab. 90: 5407-13. Epub 2005 Jun. 14). Additionally or alternatively, trophoblast-specific markers can be found using data mining tools such as the GeneCards web site (http://www.genecards.org/).

Non-limiting examples of fetal-specific hnRNA transcripts which are preferably used along with the present invention to detect fetal cell-free nuclei include the hnRNA transcripts encoding ESX1L (GenBank Accession No. NP_{—}703149), MASH2 (GenBank Accession No. NP_{—}005161), Stra 13 (GenBank Accession No. NP_{—}659435), FosB (GenBank Accession No. NP_{—}006723), Cyclin D1 (GenBank Accession No. NP_{—}444284), GCM1 (GenBank Accession No. NP_{—}003634) and Caspase-8 (GenBank Accession Nos. NP_{—}001219, NP_{—}203522, NP_{—}203521, NP 203519, NP_{—}203520).

Other suitable hnRNA transcripts which are expressed on fetal cells and can be used along with the present invention include the transcripts which encode HLA-G (GenBank Accession No. NM_{—}002127), PLAP (GenBank Accession No. NM_{—}004253), MCAM (GenBank Accession No. NM_{—}006500), laeverin (GenBank Accession No. NM_{—}173800), H315 antigen, the FT1.41.1 antigen, the NDOG-1 antigen, the NDOG-5 antigen, the BC1 antigen, the AB-154 antigen, the AB-340 antigen PAR-1 (GenBank Accession No. NM_{—}004954), Glut-12 (GenBank Accession No. NM_{—}145176), factor XIII, hPLH (GenBank Accession Nos. NM_{—}022646, NM_{—}022645, NM_{—}022644, NM_{—}020991), HLA-C (GenBank Accession No. NM_{—}002117), NDPK-A (GenBank Accession No. NM_{—}000269), Tapasin (GenBank Accession Nos. NM_{—}003190, NM_{—}172208 and NM_{—}172209), CAR (GenBank Accession No. NM_{—}001338), HASH2 (GenBank Accession No. NM_{—}005170), aHCG (GenBank Accession No. NM_{—}000735), IGF-II (GenBank Accession No. NM_{—}000612), PAI-1 (GenBank Accession No. NM_{—}000602), p57(KIP2) (GenBank Accession No. NM_{—}000076), PP5 (GenBank Accession No. NM_{—}006247), PLAC1 (GenBank Accession No. NM_{—}021796), PLAC8 (GenBank Accession No. NM_{—}016619) PLAC9 (GenBank Accession No. NM_{—}001012973), Connexin 31 (GenBank Accession No. NP_{—}076872), Connexin 43 (GenBank Accession No. NP_{—}000156), HAND 1 (GenBank Accession No. NP_{—}004812), Syncytin (GenBank Accession No. NP_{—}055405), MMP9 (GenBank Accession No. NP_{—}004985), APAF-1 (GenBank Accession Nos. NP_{—}001151, NP_{—}037361, NP_{—}863651, NP_{—}863658 and NP_{—}863659), Caspase-3 (GenBank Accession Nos. NP_{—}116786, NP_{—}004337), Caspase-9 (GenBank Accession Nos. NP_{—}127463, NP_{—}001220), FAS (GenBank Accession Nos. NP_{—}000034, NO_{—}690611, NP_{—}690610, NP_{—}690612, NP_{—}690613, NP_{—}690614, NP_{—}690616), Fas ligand (GenBank Accession No. NP_{—}000630), Flip (CASP8 and FADD-like apoptosis regulator, GenBank Accession No. NP_{—}003870), AP-2γ (GenBank Accession No. BAC11805), PLX3 (GenBank Accession No. NP_{—}004064), 313-HSD VI (GenBank Accession No. NP_{—}000853), CDX2 (GenBank Accession No. NP_{—}001256), Err2 (GenBank Accession No. NP_{—}004443), PTHrP (GenBank Accession Nos. NP_{—}002811, NP_{—}945315, NP_{—}945316 and NP_{—}945317), ASCL2/Hash2 (GenBank Accession No. NP_{—}005161), ID2 (GenBank Accession No. NP_{—}002157), HAND 1 (GenBank Accession No. NP_{—}004812), MET (GenBank Accession No. NP 000236), TEF5 (GenBank Accession No. NP_{—}003205), UPA (GenBank Accession No. NP_{—}002649), 11beta-HSD2 (GenBank Accession No. NP_{—}000187), c-Ets1 (GenBank Accession No. 1803503A), HMGI(Y) (GenBank Accession Nos. NP_{—}665906, NP_{—}002122, NP_{—}665910, NP_{—}665908, NP_{—}665909, NP_{—}665911, NP_{—}665912), estrogen receptor (GenBank Accession No. NP_{—}000116), Geml and Alpha-tocopherol transfer protein (TTPA) (GenBank Accession No. NP_{—}000361). Additional fetal-specific transcripts are described U.S. Pat. Appl. No. 20030165852 to Schueler, Paula A. et al., which is fully incorporated herein by reference and their hnRNA transcripts can be used along with the present invention.

Detection of fetal-specific nuclear RNA (e.g., H19) and/or the hnRNA transcripts according to this aspect of the present invention is preferably achieved using an RNA-in situ hybridization (RNA-ISH) staining. Preferably, such a staining utilizes polynucleotide probes capable of identifying the fetal specific nuclear RNA or the hnRNA transcripts.

As used herein the phrase “polynucleotide probe” refers to any polynucleotide which is capable of hybridizing to a target nucleic acid sequence (i.e., a nuclear RNA and/or an hnRNA transcript) present in the specimen of the present invention (e.g., the cell-free trophoblast nucleus-containing sample). Such a polynucleotide probe can be at any size, including short polynucleotides (e.g., of 15-200 bases) which correspond to small nuclear RNA or specific portions of the hnRNA transcript (e.g., intronic sequences), intermediate size polynucleotides (e.g., 100-2000 bases) and/or long polynucleotides (e.g., 2000-5000) which correspond to large portions or the complete sequence of the hnRNA transcript.

According to preferred embodiments of the present invention the polynucleotide probe used by the present invention can be any directly or indirectly labeled RNA molecule (e.g., RNA oligonucleotide, an in vitro transcribed RNA molecule), DNA molecule [e.g., oligonucleotide, complementary DNA (cDNA) molecule, genomic molecule] and/or an analogue thereof [e.g., peptide nucleic acid (PNA)] which is specific to the fetal-specific nuclear RNA and/or the hnRNA transcript of the present invention. Methods of preparing such probes are well known in the arts. Briefly, in vitro transcribed RNA probes can be generated using expression vectors and in vitro transcription kits (e.g., from Promega Corp. Madison, Wis., USA) or using PCR-generated templates essentially as described in Divjak M, et al., 2002, J. Histochem. Cytochem., 50: 541-8.

The term “oligonucleotide” as used herein refers to a single stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Preferably used oligonucleotides are those modified in the backbone, the internucleoside linkages or the bases. Non-limiting examples of modified oligonucleotides include phosphorothioates or methylated oligonucleotides. Other oligonucleotides which can be used according to the present invention are those modified in both sugar and the internucleoside linkage of the nucleotide units and are replaced with novel groups. The base units are maintained for complementation with the appropriate polynucleotide target. An example for such an oligonucleotide mimetic includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

The probes used for RNA-ISH can be directly or indirectly labeled using a tag or label molecule. Such labels can be, for example, fluorescent molecules (e.g., fluorescein or Texas Red), radioactive molecule (e.g., ³² P-γ-ATP or ³² P-α-ATP) and chromogenic substrates (e.g., Fast Red, BCIP/INT, available from ABCAM, Cambridge, Mass.). Direct labeling can be achieved by covalently conjugating to the polynucleotide (e.g., using solid-phase synthesis) or incorporating via polymerization (e.g., using an in vitro transcription reaction) the label molecule. Indirect labeling can be achieved by covalently conjugating or incorporating to the polynucleotide a non-labeled tag molecule (e.g., Digoxigenin or biotin) and subsequently subjecting the polynucleotide to a labeled molecule (e.g., anti-Digoxigenin antibody or streptavidin) capable of specifically recognizing the non-labeled tag.

RNA in situ hybridization (RNA-ISH) staining can utilize a DNA, RNA or an oligonucleotide probe which hybridizes with a specific RNA molecule (e.g., a fetal specific nuclear RNA or hnRNA transcript) present in the cell-free fetal nuclei. To prevent the RNA molecules from being degraded, the nuclei are preferably fixed to a microscopic slide using, e.g., formaldehyde or paraformaldehyde. Following fixation, a hybridization buffer containing the labeled probe (e.g., biotinylated or fluorescently labeled probe) is applied on the cell-free nuclei. The hybridization buffer includes reagents such as formamide and salts (e.g., sodium chloride and sodium citrate) which enable specific hybridization of the probe with its target nuclear RNA and/or hnRNA molecules in situ while avoiding non-specific binding of probe. Those of skills in the art are capable of adjusting the hybridization conditions (i.e., temperature, concentration of salts and formamide and the like) to specific probes and cell-free nuclei. Following hybridization, any unbound probe is washed off and the cell-free nuclei are subjected to a colorimetric reaction or a fluorescence microscope to reveal the signals generated by the bound probe. Generally, following RNA-ISH the cell-free nuclei are further counterstained as is further described hereinbelow.

The fetal nucleus-specific protein marker can be any natural peptide or polypeptide (including a glycosylated polypeptide or a proteoglycan) which is present on fetal nuclei and is absent from maternal nuclei. Non-limiting examples of fetal-specific protein markers which may be used to detect the fetal cell-free nucleus of the present invention include the ESX1L (GenBank Accession No. NP_{—}703149 Figueiredo A L, et al., 2004, J. Cell Mol. Med. 8: 545-50; Ozawa H, et al., 2004, Oncogene, 23: 6590-602), Mash2 (GenBank Accession No. NP_{—}005161), Stra 13 (GenBank Accession No. NP_{—}659435), FosB (GenBank Accession No. NP_{—}006723), Cyclin D1 (GenBank Accession No. NP_{—}444284), GCM1 (GenBank Accession No. NP_{—}003634; Baczyk D., et al., 2004, Placenta 25: 553-9; Cross JC., et al., 2003, Placenta, 24: 123-130) and Caspase-8 (GenBank Accession Nos. NP_{—}001219, NP_{—}203522, NP_{—}203521, NP_{—}203519, NP_{—}203520; De Falco M., et al., 2004, Cell Tissue Res. 318: 599-608).

Other nuclear protein markers which are expressed on fetal cells and may be used along with the present invention include MMP9 (GenBank Accession No. NP_{—}004985), APAF-1 (GenBank Accession Nos. NP_{—}001151, NP_{—}037361, NP_{—}863651, NP_{—}863658 and NP_{—}863659), Caspase-3 (GenBank Accession Nos. NP_{—}116786, NP_{—}004337), Caspase-9 (GenBank Accession Nos. NP_{—}127463, NP_{—}001220), FAS (GenBank Accession Nos. NP_{—}000034, NO_{—}690611, NP_{—}690610, NP_{—}690612, NP_{—}690613, NP 690614, NP 690616), Fas ligand (GenBank Accession No. NP_{—}000630), Flip (CASP8 and FADD-like apoptosis regulator, GenBank Accession No. NP_{—}003870), AP-2γ (GenBank Accession No. BAC11805), PLX3 (GenBank Accession No. NP_{—}004064), 3f3-HSD VI (GenBank Accession No. NP_{—}000853), CDX2 (GenBank Accession No. NP_{—}001256), Err2 (GenBank Accession No. NP_{—}004443), PTHrP (GenBank Accession Nos. NP_{—}002811, NP_{—}945315, NP_{—}945316 and NP_{—}945317; El-Hashash A H, et al., 2005, Differentiation, 73: 154-74), ASCL2/Hash2 (GenBank Accession No. NP_{—}005161; human achaete scute-like homologue 2; Zhang D, et al., 2005, Mol. Cell. Biol. 25: 6404-14), ID2 (GenBank Accession No. NP_{—}002157), HAND 1 (GenBank Accession No. NP_{—}004812), MET (GenBank Accession No. NP_{—}000236), TEF5 (GenBank Accession No. NP_{—}003205), UPA (GenBank Accession No. NP_{—}002649), 1 ibeta-HSD2 (GenBank Accession No. NP_{—}000187; Julan L., et al., 2005, Endocrinology, 146: 1482-90), c-Ets1 (GenBank Accession No. 1803503A; Takai N, et al., Gynecol Obstet. Invest. 2005, 61: 15-20), HMGI(Y) (GenBank Accession Nos. NP_{—}665906, NP_{—}002122, NP_{—}665910, NP_{—}665908, NP_{—}665909, NP_{—}665911, NP_{—}665912; Bamberger A M, et al., 2003, Virchows Arch. 443: 649-54), estrogen receptor (GenBank Accession No. NP_{—}000116; Billiar R B, et al., 1997, Placenta, 18: 365-70), Alpha-tocopherol transfer protein (TTPA) (GenBank Accession No. NP_{—}000361; Muller-Schmehl K, et al., 2004, Free Radic Res. 38: 413-20), as well as proteins of the nuclear envelope and other proteins described in Morrish D. W., et al., 2001 (Current Protein and Peptide Science, 2: 245-259).

Identification of the fetal-nucleus specific protein marker of the present invention may be performed using an immunological staining and a labeled antibody directed against the fetal nucleus-specific protein marker.

Antibodies directed against fetal nucleus-specific protein marker are known in the art and include, for example, the MMP9 (2C3) (monoclonal IgG, SC-21733, Santa Cruz Biotechnology, Inc., Santa Cruz, Calif., USA), Fos-b (C-11) (monoclonal IgM, SC-8013, Santa Cruz), C-Jun (Novus Biologicals, Littleton, Colo.), Fra-2 (Q-20) (SC-604, Santa Cruz), HAND 1 (Abcam) and UPA (Abcam).

Immunological staining is based on the binding of labeled antibodies to antigens present on cell compartment (e.g., the nucleus). It will be appreciated that the labeled antibodies can be either primary antibodies (i.e., which bind to the specific antigen, e.g., fetal nucleus-specific protein marker) or secondary antibodies (e.g., labeled goat anti rabbit antibodies, labeled mouse anti human antibody) which bind to the primary antibodies. An immunological staining can be achieved using an antibody which is directly conjugated to a label or is a conjugated enzyme. Examples of immunological staining procedures include but are not limited to, fluorescently labeled immunohistochemistry (using a fluorescent dye conjugated to an antibody), radiolabeled immunohistochemistry (using radiolabeled e.g., ¹²⁵I, antibodies), and immunocytochemistry [using an enzyme (e.g., horseradish peroxidase or alkaline phosphatase) and a chromogenic substrate to produce a colorimetric reaction]. It will be appreciated that the enzymes conjugated to antibodies can utilize various chromogenic substrates such as AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate (Avivi C., et al., 1994, J Histochem. Cytochem. 1994; 42: 551-4) for alkaline phosphatase enzyme and Nova Red, diaminobenzidine (DAB), Vector(R) SG substrate, luminol-based chemiluminescent substrate for the peroxidase enzyme. These enzymatic substrates are commercially available from Sigma (St Louis, Mo., USA), Molecular Probes Inc. (Eugene, Oreg., USA), Vector Laboratories Inc. (Burlingame, Calif., USA), Zymed Laboratories Inc. (San Francisco, Calif., USA), Dako Cytomation (Denmark).

The immunological staining may be immunohistochemistry and/or immunocytochemistry.

It will be appreciated that immunological staining of cell-free fetal nuclei can be performed by adjusting a protocol suitable for whole cells, except that the antibodies used are directed against the fetal nucleus-specific protein marker. Briefly, to detect a fetal-nucleus specific protein marker in a transcervical specimen, cytospin slides are washed in 70% alcohol solution and dipped for 5 minutes in distilled water. The slides are then transferred into a moist chamber, washed three times with phosphate buffered-saline (PBS). To visualize the position of the cell-free fetal nuclei on the microscopic slides, the borders of the transcervical specimens are marked using e.g., a Pap Pen (Zymed Laboratories Inc., San Francisco, Calif., USA). To block endogenous peroxidase activity, 50-100 μl of a 3% hydrogen peroxide (Merck, Germany) solution are added to each slide for 5-10 minutes incubation at room temperature following which the slides are washed three times in PBS. To avoid non-specific binding of the antibody, two drops of a blocking reagent (e.g., Zymed HISTOSTAIN®-PLUS Kit, Cat No. 858943) are added to each slide for 5-10 minutes incubation in a moist chamber. To identify the cell-free fetal nuclei in the transcervical sample, an aliquot (e.g., 50 μl) of a fetal-nucleus specific protein marker antibody [e.g., anti MMP9 or anti Fos-b] is added to the slides. The slides are then incubated with the antibody in a moist chamber for 60 minutes, following which they are washed three times with PBS. To detect the bound primary antibody, two drops of a secondary biotinylated antibody (e.g., goat anti-mouse IgG antibody available from Zymed) are added to each slide for 10-15 minutes incubation in a moist chamber. The secondary antibody is washed off three times with PBS. To reveal the biotinylated secondary antibody, two drops of an horseradish peroxidase (HRP)-streptavidin conjugate (available from Zymed) are added for 10-15 minutes incubation in a moist chamber, followed by three washes in PBS. Finally, to detect the HRP-conjugated streptavidin, two drops of an aminoethylcarbazole (AEC Single Solution Chromogen/Substrate, Zymed) HRP substrate are added for 6-10 minutes incubation in a moist chamber, followed by three washed with PBS. It will be appreciated that following an immunological staining the nuclei can be counterstained with a cytological stain such as May-Grünwald-Giemsa stain, Giemsa stain, Papanicolau stain, Hematoxylin stain and DAPI stain. For example, following immunological staining with the fetal nucleus specific antibody, counterstaining can be performed by applying for 15 seconds 2 drops of 0.2% of Hematoxylin solution (e.g., Sigma-Aldrich Corp., St Louis, Mo., USA, Cat. No. GHS-2-32). The slides are then washed under tap water and covered with a coverslip.

An epigenetic marker can be detected in the cell-free fetal nucleus by combining fluorescent immunostaining with an antibody directed against 5-methylcytosine (5MeC) (Reynaud et al.: Cancer Lett. 61:255-262, 1991) and an in situ hybridization with a beta-satellite DNA probe, essentially as described in de Capoa A, et al., Cytometry, 1998, 31: 85-92, which is fully incorporated herein by reference.

It will be appreciated that once the fetal cell-free nucleus is identified it can be subject to molecular analysis enabling prenatal diagnosis of the fetus. This includes determination of fetal gender and/or paternity and identification of chromosomal, or DNA abnormalities. Such analysis, which uses far higher numbers of fetal genetic and protein material as compared with prior art approaches, present a new and robust method of non-invasive prenatal diagnosis.

As is shown in the Figures and is described in the Examples section which follows, the cell-free fetal nuclei exhibit clear FISH signals which can easily identify fetal gender and/or chromosomal abnormalities.

Thus, according to another aspect of the present invention, there is provided a method of analyzing a genetic material of a fetus. The method is achieved by (a) detecting a cell-free nucleus in a sample; and/or (b) detecting in a cell-free nucleus at least one fetal-nucleus specific marker; thereby identifying a fetal nucleus; and (c) molecularly analyzing the genetic material in the fetal nucleus, thereby analyzing the genetic material of the fetus.

As used herein, the phrase “genetic material” refers to the DNA, RNA or protein present in cells of the fetus.

The phrase “analyzing a genetic material of a fetus” refers to the identification of any genetic characteristic of the fetus using the DNA, RNA or protein derived from the fetus. For example, analysis of the genetic material of the fetus according to this aspect of the present invention may refer to determining the presence or absence of at least one chromosomal abnormality, determining the presence or the absence of at least one DNA abnormality, determining a paternity of the fetus, determining the gender of the fetus, determining the presence or absence of a specific polymorphic allele, and/or analyzing the genetic makeup of the fetus. Analysis of the expression level and/or activity of specific fetal RNA and/or proteins can be performed using methods know in the art (e.g., RNA-ISH, immunostaining and activity staining) as described hereinabove and in US20050181429A1, which is fully incorporated herein by reference.

As used herein “gender of a fetus” refers to the presence or absence of the X and/or Y chromosome(s) in the fetus.

To identify or diagnose chromosomal and/or DNA abnormalities and/or fetal paternity or gender, the identified cell-free fetal nucleus or nuclei of the present invention are preferably subjected to a molecular analysis of the genetic material.

Preferably, such a molecular analysis utilizes an approach such as in situ chromosomal analysis, in situ DNA analysis and/or genetic analysis.

The phrase “in situ chromosomal analysis” refers to the analysis of the chromosome(s) within the cell-free nucleus, using fluorescent in situ hybridization (FISH), and/or multicolor-banding (MCB).

Methods of employing FISH analysis on interphase chromosomes are known in the art. Briefly, directly-labeled probes [e.g., the CEP X green and Y orange (Abbott cat no. 5J10-51)] are mixed with hybridization buffer (e.g., LSI/WCP, Abbott) and a carrier DNA (e.g., human Cot 1 DNA, available from Abbott). The probe solution is applied on microscopic slides containing e.g., transcervical cytospin specimens and the slides are covered using coverslips. The probe-containing slides are denatured for 4-5 minutes at 71° C. (or 3 minutes at 70° C.) and are further incubated for 24-60 hours at 37° C. using an hybridization apparatus (e.g., HYBrite, Abbott Cat. No. 2J11-04). It is worth mentioning that although the manufacturer (Abbott) recommends hybridization for 48 hours at 37° C., similar results can be obtained if hybridization lasts for 60 hours. According to presently preferred configurations, hybridization takes place between 48-60 hours at 37° C. To increase the specificity, following hybridization, the slides are washed for 2 minutes at 70-72° C. in a solution of 0.3% NP-40 (Abbott) in 60 mM NaCl and 6 mM NaCltrate (0.4×SSC). Slides are then immersed for 1 minute in a solution of 0.1% NP-40 in 2×SSC at room temperature, following which the slides are allowed to dry in the dark. Counterstaining is performed using, for example, DAPI II counterstaining (Abbott).

High-resolution multicolor banding (MCB) on interphase chromosomes, which is described in detail by Lemke et al. (Am. J. Hum. Genet. 71: 1051-1059, 2002) uses YAC/BAC and region-specific microdissection DNA libraries as DNA probes for interphase chromosomes. Briefly, for each region-specific DNA library 8-10 chromosome fragments are excised using microdissection and the DNA is amplified using a degenerated oligonucleotide PCR reaction. For example, for MCB staining of chromosome 5, seven overlapping microdissection DNA libraries were constructed, two within the p arm and five within the q arm (Chudoba I., et al., 1999; Cytogenet. Cell Genet. 84: 156-160). Each of the DNA libraries is labeled with a unique combination of fluorochromes and hybridization and post-hybridization washes are carried out using standard protocols (see for example, Senger et al., 1993; Cytogenet. Cell Genet. 64: 49-53). Analysis of the multicolor-banding can be performed using the isis/mFISH imaging system (MetaSystems GmbH, Altlussheim, Germany). It will be appreciated that although MCB staining on interphase chromosomes was documented for a single chromosome at a time, it is conceivable that additional probes and unique combinations of fluorochromes can be used for MCB staining of two or more chromosomes at a single MCB analysis. Thus, this technique can be used along with the present invention to identify fetal chromosomal aberrations, particularly, for the detection of specific chromosomal abnormalities which are known to be present in other family members.

The phrase “in situ DNA analysis” refers to DNA-based analysis (e.g., primer extension) which is performed on the cell-free fetal nucleus such as primed in situ labeling (PRINS) or quantitative FISH (Q-FISH).

Methods of performing PRINS analysis are known in the art and include for example, those described in Coullin, P. et al. (Am. J. Med. Genet. 2002, 107: 127-135); Findlay, I., et al. (J. Assist. Reprod. Genet. 1998, 15: 258-265); Musio, A., et al. (Genome 1998, 41: 739-741); Mennicke, K., et al. (Fetal Diagn. Ther. 2003, 18: 114-121); Orsetti, B., et al. (Prenat. Diagn. 1998, 18: 1014-1022). Briefly, slides containing interphase chromosomes are denatured for 2 minutes at 71° C. in a solution of 70% formamide in 2×SSC (pH 7.2), dehydrated in an ethanol series (70, 80, 90 and 100%) and are placed on a flat plate block of a programmable temperature cycler (such as the PTC-200 thermal cycler adapted for glass slides which is available from MJ Research, Waltham, Mass., USA). The PRINS reaction is usually performed in the presence of unlabeled primers and a mixture of dNTPs with a labeled dUTP (e.g., fluorescein-12-dUTP or digoxigenin-11-dUTP for a direct or indirect detection, respectively). Alternatively, or additionally, the sequence-specific primers can be labeled at the 5′ end using e.g., 1-3 fluorescein or cyanine 3 (Cy3) molecules. Thus, a typical PRINS reaction mixture includes sequence-specific primers (50-200 μmol in a 50 μl reaction volume), unlabeled dNTPs (0.1 mM of dATP, dCTP, dGTP and 0.002 mM of dTTP), labeled dUTP (0.025 mM) and Taq DNA polymerase (2 units) with the appropriate reaction buffer. Once the slide reaches the desired annealing temperature the reaction mixture is applied on the slide and the slide is covered using a cover slip. Annealing of the sequence-specific primers is allowed to occur for 15 minutes, following which the primed chains are elongated at 72° C. for another 15 minutes. Following elongation, the slides are washed three times at room temperature in a solution of 4×SSC/0.5% Tween-20 (4 minutes each), followed by a 4-minute wash at PBS. Slides are then subjected to nuclei counterstaining using DAPI or propidium iodide. The fluorescently stained slides can be viewed using a fluorescent microscope and the appropriate combination of filters (e.g., DAPI, FITC, TRITC, FITC-rhodamin).

It will be appreciated that several primers which are specific for several targets can be used on the same PRINS run using different 5′ conjugates. Thus, the PRINS analysis can be used as a multicolor assay for the determination of the presence, and/or location of several genes or chromosomal loci. Thus, PRINS analysis has been employed in the detection of gene deletion (Tharapel S A and Kadandale J S, 2002. Am. J. Med. Genet. 107: 123-126), determination of fetal sex (Orsetti, B., et al., 1998. Prenat. Diagn. 18: 1014-1022), and identification of chromosomal aneuploidy (Mennicke, K. et al., 2003. Fetal Diagn. Ther. 18: 114-121). In addition, as described in Coullin et al., (2002, Supra) the PRINS analysis can be performed on the same slide as the FISH analysis, preferably, prior to FISH analysis.

Quantitative FISH (Q-FISH) can be used to detect chromosomal abnormalities by measuring variations in fluorescence intensity of specific probes which hybridize to chromosomal DNA. Q-FISH can be performed using Peptide Nucleic Acid (PNA) oligonucleotide probes as described hereinabove. The hydrophobic and neutral backbone of PNA probes enables high affinity and specific hybridization to the nucleic acid counterparts (e.g., chromosomal DNA) (Pellestor F and Paulasova P, 2004; Chromosoma 112: 375-380). Q-FISH has been applied on interphase nuclei to monitor telomere stability (Slijepcevic, P. 1998; Mutat. Res. 404:215-220; Henderson S., et al., 1996; J. Cell Biol. 134: 1-12), the presence of Fanconi aneamia (Hanson H, et al., 2001, Cytogenet. Cell Genet. 93: 203-6) and numerical chromosome abnormalities such as trisomy 18 (Chen C, et al., 2000, Mamm. Genome 10: 13-18), as well as monosomy, duplication, and deletion (Taneja K L, et al., 2001, Genes Chromosomes Cancer. 30: 57-63).

Alternatively, Q-FISH can be performed by co-hybridizing whole chromosome painting probes (e.g., for chromosomes 21 and 22) on interphase nuclei as described in Truong K et al, 2003, Prenat. Diagn. 23: 146-51.

Since the in situ chromosomal and/or DNA analysis is performed on the same cell-free fetal nucleus, the method according to this aspect of the present invention can diagnose the fetus, i.e., determine fetal gender and/or paternity and identify at least one chromosomal and/or DNA abnormality of the fetus.

As used herein, the phrase “chromosomal abnormality” refers to an abnormal number of chromosomes (e.g., trisomy 21, monosomy X) or to chromosomal structure abnormalities (e.g., deletions, translocations, etc).

According to preferred embodiments of the present invention, the chromosomal abnormality can be chromosomal aneuploidy (i.e., complete and/or partial trisomy and/or monosomy), translocation, subtelomeric rearrangement, deletion, microdeletion, inversion and/or duplication (i.e., complete an/or partial chromosome duplication).

According to preferred embodiments of the present invention the trisomy detected by the present invention can be trisomy 21 [using e.g., the LSI 21q22 orange labeled probe (Abbott cat no. 5J13-02)], trisomy 18 [using e.g., the CEP 18 green labeled probe (Abbott Cat No. 5J10-18); the CEP®18 (D18Z1, ac satellite) Spectrum Orange™ probe (Abbott Cat No. 5J08-18)], trisomy 16 [using e.g., the CEP16 probe (Abbott Cat. No. 6J37-17)], trisomy 13 [using e.g., the LSI® 13 SpectrumGreen™ probe (Abbott Cat. No. 5J14-18)], and the XXY, XYY, or XXX trisomies which can be detected using e.g., the CEP X green and Y orange probe (Abbott cat no. 5J10-51); and/or the CEP®X SpectrumGreen™/CEP® Y (μ satellite) SpectrumOrange™ probe (Abbott Cat. No. 5J10-51).

It will be appreciated that using the chromosome-specific FISH probes, PRINS primers, Q-FISH and MCB staining various other trisomies and partial trisomies can be detected in fetal cells according to the teachings of the present invention. These include, but not limited to, partial trisomy 1q32-44 (Kimya Y et al., Prenat Diagn. 2002, 22:957-61), trisomy 9 p with trisomy 10p (Hengstschlager M et al., Fetal Diagn Ther. 2002, 17:243-6), trisomy 4 mosaicism (Zaslav A L et al., Am J Med. Genet. 2000, 95:381-4), trisomy 17p (De Pater J M et al., Genet Couns. 2000, 11:241-7), partial trisomy 4q26-qter (Petek E et al., Prenat Diagn. 2000, 20:349-52), trisomy 9 (Van den Berg C et al., Prenat. Diagn. 1997, 17:933-40), partial 2p trisomy (Siffroi J P et al., Prenat Diagn. 1994, 14:1097-9), partial trisomy 1q (DuPont B R et al., Am J Med. Genet. 1994, 50:21-7), and/or partial trisomy 6p/monosomy 6q (Wauters J G et al., Clin Genet. 1993, 44:262-9).

The method of the present invention can be also used to detect several chromosomal monosomies such as, monosomy 22, 16, 21 and 15, which are known to be involved in pregnancy miscarriage (Munne, S. et al., 2004. Reprod Biomed Online. 8: 81-90)].

According to preferred embodiments of the present invention the monosomy detected by the method of the present invention can be monosomy X, monosomy 21, monosomy 22 [using e.g., the LSI 22 (BCR) probe (Abbott, Cat. No. 5J17-24)], monosomy 16 (using e.g., the CEP 16 (D16Z3) Abbott, Cat. No. 6J36-17) and monosomy 15 [using e.g., the CEP 15 (D15Z4) probe (Abbott, Cat. No. 6J36-15)].

It will be appreciated that several translocations and microdeletions can be asymptomatic in the carrier parent, yet can cause a major genetic disease in the offspring. For example, a healthy mother who carries the 15q11-q13 microdeletion can give birth to a child with Angelman syndrome, a severe neurodegenerative disorder. Thus, the present invention can be used to identify such a deletion in the fetus using e.g., FISH probes which are specific for such a deletion (Erdel M et al., Hum Genet. 1996, 97: 784-93).

Thus, the present invention can be also used to detect any chromosomal abnormality if one of the parents is a known carrier of such abnormality. These include, but not limited to, mosaic for a small supernumerary marker chromosome (SMC) (Giardino D et al., Am J Med. Genet. 2002, 111:319-23); t(11;14)(p15;p13) translocation (Benzacken B et al., Prenat Diagn. 2001, 21:96-8); unbalanced translocation t(8;11)(p23.2;p15.5) (Fert-Ferrer S et al., Prenat Diagn. 2000, 20:511-5); 11q23 microdeletion (Matsubara K, Yura K. Rinsho Ketsueki. 2004, 45:61-5); Smith-Magenis syndrome 17p11.2 deletion (Potocki L et al., Genet Med. 2003, 5:430-4); 22q13.3 deletion (Chen CP et al., Prenat Diagn. 2003, 23:504-8); Xp22.3. microdeletion (Enright F et al., Pediatr Dermatol. 2003, 20:153-7); 10p14 deletion (Bartsch O, et al., Am J Med. Genet. 2003, 117A:1-5); 20p microdeletion (Laufer-Cahana A, Am J Med. Genet. 2002, 112:190-3.), DiGeorge syndrome [del(22)(q11.2q11.23)], Williams syndrome [7q11.23 and 7q36 deletions, Wouters C H, et al., Am J Med. Genet. 2001, 102:261-5.]; 1p36 deletion (Zenker M, et al., Clin Dysmorphol. 2002, 11:43-8); 2p microdeletion (Dee S L et al., J Med. Genet. 2001, 38:E32); neurofibromatosis type 1 (17q11.2 microdeletion, Jenne D E, et al., Am J Hum Genet. 2001, 69:516-27); Yq deletion (Toth A, et al., Prenat Diagn. 2001, 21:253-5); Wolf-Hirschhorn syndrome (WHS, 4p16.3 microdeletion, Rauch A et al., Am J Med. Genet. 2001, 99:338-42); 1p36.2 microdeletion (Finelli P, Am J Med Genet. 2001, 99:308-13); 11q14 deletion (Coupry I et al., J Med. Genet. 2001, 38:35-8); 19q13.2 microdeletion (Tentler D et al., J Med. Genet. 2000, 37:128-31); Rubinstein-Taybi (16 p13.3 microdeletion, Blough R1, et al., Am J Med. Genet. 2000, 90:29-34); 7p21 microdeletion (Johnson D et al., Am J Hum Genet. 1998, 63:1282-93); Miller-Dieker syndrome (17p13.3), 17p11.2 deletion (Juyal R C et al., Am J Hum Genet. 1996, 58:998-1007); 2q37 microdeletion (Wilson L C et al., Am J Hum Genet. 1995, 56:400-7).

The present invention can be used to detect inversions [e.g., inverted chromosome X (Lepretre, F. et al., Cytogenet. Genome Res. 2003. 101: 124-129; Xu, W. et al., Am. J. Med. Genet. 2003. 120A: 434-436), inverted chromosome 10 (Helszer, Z., et al., 2003. J. Appl. Genet. 44: 225-229)], cryptic subtelomeric chromosome rearrangements (Engels, H., et al., 2003. Eur. J. Hum. Genet. 11: 643-651; Bocian, E., et al., 2004. Med. Sci. Monit. 10: CR143—CR151), and/or duplications (Soler, A., et al., Prenat. Diagn. 2003. 23: 319-322).

It will be appreciated that once the fetal nucleus is identified, it is preferably photographed using e.g., a CCD camera. In order to subject the same cell-free fetal nuclei to further chromosomal and/or DNA analysis, the position (i.e., coordinate location) of such cell-free fetal nuclei on the slide is stored in the microscope or the computer connected thereto for later reference. Examples of microscope systems which enable identification and storage of nuclei coordinates include the Bio View Duet™ (Bio View Ltd., Rehovot, Israel), and the Applied Imaging System (Newcastle England), essentially as described in Merchant, F. A. and Castleman K. R. (Hum. Repr. Update, 2002, 8: 509-521).

In addition, in order to make the stained cell-free nuclei amenable to a subsequent in situ chromosomal and/or DNA analysis as is further described hereinbelow, the stained cell-free fetal nuclei are preferably subject to an additional treatment which removes residual dyes of previous stains (e.g., AEC, Fast red, hematoxylin). Such a treatment may include for example, washing off the bound antibody (using e.g., water and a gradual ethanol series), exposing cell nuclei (using e.g., a methanol-acetic acid fixer), digesting proteins (using e.g., Pepsin), and immersing the slides in a solution of 2% ammonium hydroxide (diluted in 70% alcohol) for an incubation of about 45 minutes followed by a 5-10 minutes wash in distilled water.

Thus, the teachings of the present invention can be used to identify chromosomal aberrations in a fetus without subjecting the mother to invasive and risk-carrying procedures.

For example, in order to determine fetal gender and/or the presence of a Down syndrome fetus (i.e., trisomy 21) according to the teachings of the present invention, a transcervical specimen is obtained from a pregnant woman at 5^{th} to the 15^{th} weeks of gestation using a Pap smear cytobrush. The cell free-nuclei are suspended in RPMI-1640 medium tissue culture medium (Beth Haemek, Israel) in the presence of 1% Penicillin Streptomycin antibiotic, and cytospin slides are prepared using a Cytofunnel Chamber Cytocentrifuge (Thermo-Shandon, England) according to manufacturer's instructions. Cytospin slides are dehydrated in 95% alcohol until immunohistochemical analysis is performed.

Prior to immunohistochemistry, cytospin slides are hydrated in 70% alcohol and water, washed with PBS, treated with 3% hydrogen peroxide followed by three washes in PBS and incubated with a blocking reagent (from the Zymed HISTOSTAIN®-PLUS Kit, Cat No. 858943). An MMP9 antibody is applied on the slides according to manufacturer's instructions for a 60-minutes incubation followed by 3 washes in PBS. A secondary biotinylated goat anti-mouse IgG antibody (Zymed HISTOSTAIN®-PLUS Kit, Cat No. 858943) is added to the slide for a 10-minute incubation followed by three washes in PBS. The secondary antibody is then retrieved using the HRP-streptavidin conjugate (Zymed HISTOSTAIN®-PLUS Kit, Cat No. 858943) and the aminoethylcarbazole (AEC Single Solution Chromogen/Substrate, Zymed) HRP substrate according to manufacturer's instructions. Counterstaining is performed using a diluted solution (e.g., 0.2-1%) of Hematoxylin (Sigma-Aldrich Corp., St Louis, Mo., USA, Cat. No. GHS-2-32). The immunologically stained cell-free fetal nuclei are viewed and photographed using a light microscope (Olympus BX61, Olympus, Japan) and a CCD camera (Applied Imaging, Newcastle, England) connected thereto. The microscope coordinates of the identified MMP9-positive cell-free fetal nuclei are marked and stored.

To remove antibody's residual staining, stained slides are washed for 5-10 minutes in water, immersed for 45 minutes in 2% ammonium hydroxide (diluted in 70% alcohol) and washed for 5-10 minutes in distilled water. Prior to FISH analysis slides containing e.g., Fos-b—positive cell-free fetal nuclei are dehydrated in 70% and 100% ethanol, and fixed for 45 minutes in an ice-cold methanol-acetic acid (in a 3:1 ratio) fixer solution. Slides are then washed in a warm solution (at 37° C.) of 2×SSC, fixed in 0.9% of formaldehyde in PBS and washed in PBS. Prior to FISH analysis, slides are digested with a Pepsin solution (0.15% in 0.01 N HCl), dehydrated in an ethanol series and dried.

For the determination of fetal gender, 7 μl of the LSI/WCP hybridization buffer (Abbott) are mixed with 1 μl of the directly-labeled CEP X green and Y orange probes containing the centromere regions Xp11.1-q11.1 (DXZ1) and Ypl1.1-q11.1 (DYZ3) (Abbott Cat no. 5J10-51), 1-2 μl of human Cot 1 DNA (1 μg/μl, Abbott, Cat No. 06J31-001) and 2 μl of purified double-distilled water. The probe-hybridization solution is centrifuged for 1-3 seconds and 11 μl of the probe-hybridization solution is applied on each slide, following which, the slides are immediately covered using a coverslip. Slides are then denatured for 4-5 minutes at 71° C. and further incubated at 37° C. for 24-60 hours (e.g., for 48-60 hours) in the HYBrite apparatus (Abbott Cat. No. 2J11-04). Following hybridization, slides are washed in 0.3% NP-40 in 0.4×SSC, followed by 0.1% NP-40 in 2×SSC and are allowed to dry in the dark. Counterstaining is performed using DAPI II (Abbott). Slides are then viewed using a fluorescent microscope (BX61, Olympus, Japan) according to the previously marked positions of the Fos-b—positive cell-free fetal nuclei and photographed.

For the determination of the presence or absence of a Down syndrome fetus, following the first set of FISH analysis the slides are washed in 1×SSC (20 minutes, room temperature) following which they are dipped for 10 seconds in purified double-distilled water at 71° C. Slides are then dehydrated in an ethanol series and dried. Hybridization is achieved using the LSI 21q22 orange labeled probe containing the D21S259, D21S341 and D21S342 loci within the 21q22.13 to 21q22.2 region (Abbott cat no. 5J13-02) and the same hybridization and washing conditions as used for the first set of FISH probes. The FISH signals obtained following the second set of FISH probes are viewed using the fluorescent microscope and the same coordination of Fos-b positive cell-free fetal nuclei.

The use of FISH probes for chromosomes 13, 18, 21, X and Y on interphase chromosomes was found to reduce the residual risk for a clinically significant abnormality from 0.9-10.1% prior to the interphase FISH assay, to 0.6-1.5% following a normal interphase FISH pattern [Horner J, et al., 2003. Residual risk for cytogenetic abnormalities after prenatal diagnosis by interphase fluorescence in situ hybridization (FISH). Prenat Diagn. 23: 566-71]. Thus, the teachings of the present invention can be used to significantly reduce the risk of having clinically abnormal babies by providing an efficient method of prenatal diagnosis.

Alternatively, in situ chromosomal and/or DNA analysis can be performed on cell-free fetal nuclei previously subjected to an RNA-ISH staining with a probe specific to a fetal-nucleus marker such as the H19 transcript (SEQ ID NO:1). The cell-free nuclei are preferably fixed (using, for example, a methanol-acetic acid fixer solution) and treated with an enzyme such as Pepsin, which is capable of degrading all nuclear structures. Those of skills in the art are capable of adjusting various treatment protocols (i.e., fixation and digestion) according to the cell-free nuclei or the probes used.

The signal obtained using the RNA-ISH probe can be developed prior to the in situ chromosomal staining (e.g., in case of FISH or Q-FISH are utilized) or simultaneously with the in situ chromosomal staining (e.g., in case a biotinylated probe is utilized for the RNA-ISH staining and a directly labeled fluorescent probe is employed for the FISH analysis). Preferably, following RNA-ISH the stained cell-free fetal nuclei are counterstained and further subjected to in situ chromosomal and/or DNA analysis.

As is mentioned hereinabove, the method according to this aspect of the present invention can be also used to diagnose at least one DNA abnormality in the fetus.

The phrase “DNA abnormality” refers to a single nucleotide substitution, deletion, insertion, micro-deletion, micro-insertion, short deletion, short insertion, multinucleotide substitution, and abnormal DNA methylation and loss of imprint (LOI). Such a DNA abnormality can be related to an inherited genetic disease such as a single-gene disorder (e.g., cystic fibrosis, Canavan, Tay-Sachs disease, Gaucher disease, Familial Dysautonomia, Niemann-Pick disease, Fanconi anemia, Ataxia telaugiestasia, Bloom syndrome, Familial Mediterranean fever (FMF), X-linked spondyloepiphyseal dysplasia tarda, factor XI), an imprinting disorder [e.g., Angelman Syndrome, Prader-Willi Syndrome, Beckwith-Wiedemann syndrome, Myoclonus-dystonia syndrome (MDS)], predisposition to various cancer diseases (e.g., mutations in the BRCA1 and BRCA2 genes), as well as disorders which are caused by minor chromosomal aberrations (e.g., minor trisomy mosaicisms, duplication sub-telomeric regions, interstitial deletions or duplications) which are below the detection level of conventional in situ chromosomal and/or DNA analysis methods (i.e., FISH, Q-FISH, MCB and PRINS).

According to preferred embodiments of this aspect of the present invention identification of at least one DNA abnormality is achieved by a genetic analysis.

The phrase “genetic analysis” as used herein refers to any chromosomal, DNA and/or RNA-based analysis which can detect chromosomal, DNA and/or gene expression abnormalities, respectively in a cell-free nucleus of the fetus.

As is mentioned hereinabove, major and minor chromosomal abnormalities can be detected in interphase chromosomes using conventional methods such as FISH, Q-FISH, MCB and PRINS. However, the identification of some subtle chromosomal abnormalities require the application of DNA-based detection methods such as comparative genome hybridization (CGH).

Comparative Genome Hybridization (CGH) is based on a quantitative two-color fluorescence in situ hybridization (FISH) on metaphase chromosomes. In this method a test DNA (e.g., DNA extracted from the cell-free fetal nucleus of the present invention) is labeled in one color (e.g., green) and mixed in a 1:1 ratio with a reference DNA (e.g., DNA extracted from a control cell-free nucleus) which is labeled in a different color (e.g., red). Methods of amplifying and labeling whole-genome DNA are well known in the art (see for example, Wells D, et al., 1999; Nucleic Acids Res. 27: 1214-8). Briefly, genomic DNA is amplified using a degenerate oligonucleotide primer [e.g., 5′-CCGACTCGAGNNNNNNATGTGG, SEQ ID NO:2 (Telenius, H., et al., 1992; Genomics 13:718-25)] and the amplified DNA is labeled using e.g., the Spectrum Green-dUTP (for the test DNA) or the Spectrum Red-dUTP (for the reference DNA). The mixture of labeled DNA samples is precipitated with Cotl DNA (Gibco-BRL) and resuspended in an hybridization mixture containing e.g., 50% formamide, 2×SSC, pH 7 and 10% dextrane sulfate.

Prior to hybridization, the labeled DNA samples (i.e., the probes) are denatured for 10 minutes at 75° C. and allowed to cool at room temperature for 2 minutes. Likewise, the metaphase chromosome spreads are denatured using standard protocols (e.g., dehydration in a series of ethanol, denaturation for 5 minutes at 75° C. in 70% formamide and 2×SSC). Hybridization conditions include incubation at 37° C. for 25-hours in a humidified chamber, following by washes in 2×SSC and dehydration using an ethanol series, essentially as described elsewhere (Wells, D., et al., 2002; Fertility and Sterility, 78: 543-549). Hybridization signal is detected using a fluorescence microscope and the ratio of the green-to-red fluorescence can be determined using e.g., the Applied Imaging (Santa Clara, Calif.) computer software. If both genomes are equally represented in the metaphase chromosomes (i.e., no deletions, duplication or insertions in the DNA derived from the trophoblast nucleus) the labeling on the metaphase chromosomes is orange. However, regions which are either deleted or duplicated in the cell-free fetal nucleus are stained with red or green, respectively.

It will be appreciated that since the cell-free fetal nucleus of the present invention is processed according to the method of the present invention to include interphase chromosomes, the metaphase chromosomes used by the CGH method are derived from the reference cell-free nucleus (i.e., of a normal individual) having a karyotype of either 46, XY or 46, XX.

DNA array-based comparative genomic hybridization (CGH-array), which is fully described in Hu, D. G., et al., 2004, Mol. Hum. Reprod. 10: 283-289, is a modified version of CGH and is based on the hybridization of a 1:1 mixture of the test and reference DNA probes on an array containing chromosome-specific DNA libraries. Methods of preparing chromosome-specific DNA libraries are known in the art (see for example, Bolzer A., et al., 1999; Cytogenet. Cell. Genet. 84: 233-240). Briefly, single chromosomes are obtained using either microdissection or flow-sorting and the genomic DNA of each of the isolated chromosomes is PCR-amplified using a degenerated oligonucleotide primer. To remove repetitive DNA sequences, the amplified DNA is subjected to affinity chromatography in combination with negative subtraction hybridization (using e.g., human Cot-1 DNA or centromere-specific repetitive sequence as subtractors), essentially as described in Craig J M., et al., 1997; Hum. Genet. 100: 472-476. Amplified chromosome-specific DNA libraries are then attached to a solid support [(e.g., SuperAmine slides (TeleChem, USA)], dried, baked and washed according to manufacturer's recommendation. Labeled genomic DNA probes (a 1:1 mixture of the test and reference DNAs) are mixed with non-specific carrier DNA (e.g., human Cot-1 and/or salmon sperm DNA, Gibco-BRL), ethanol-precipitated and re-suspended in an hybridization buffer such as 50% deionized formamide, 2×SSC, 0.1% SDS, 10% Dextran sulphate and 5×Denhardt's solution. The DNA probes are then denatured (80° C. for 10 minutes), pre-annealed (37° C. for 80 minutes) and applied on the array for hybridization of 15-20 hours in a humid incubator. Following hybridization the arrays are washed twice for 10 minutes in 50% formamide/2×SSC at 45° C. and once for 10 minutes in 1×SSC at room temperature, following which the arrays are rinsed three times in 18.2 MΩ deionized water. The arrays are then scanned using any suitable fluorescence scanner such as the GenePix 4000B microarray reader (Axon Instruments, USA) and analyzed using the GenePix Pro. 4.0.1.12 software (Axon).

The DNA-based CGH-array technology was shown to confirm fetal abnormalities detected using conventional G-banding and to identify additional fetal abnormalities such as mosaicism of trisomy 20, duplication of 10q telomere region, interstitial deletion of chromosome 9p and interstitial duplication of the PWS region on chromosome 15q which is implicated in autism if maternally inherited (Schaeffer, A. J., et al., 2004; Am. J. Hum. Genet. 74: 1168-1174), unbalanced translocation (Klein O D, et al., 2004, Clin Genet. 65: 477-82), unbalanced subtelomeric rearrangements (Ness G O et al., 2002, Am. J. Med. Genet. 113: 125-36), unbalanced inversions and/or chromosomal rearrangements (Daniely M, et al., 1999; Cytogenet Cell Genet. 86: 51-5).

The identification of single gene disorders, imprinting disorders, and/or predisposition to cancer can be achieved using any method suitable for identification of at least one nucleic acid substitution such as a single nucleotide polymorphism (SNP).

Direct sequencing of a PCR product is based on the amplification of a genomic sequence using specific PCR primers in a PCR reaction following by a sequencing reaction utilizing the sequence of one of the PCR primers as a sequencing primer. Sequencing reaction can be performed using, for example, the Applied Biosystems (Foster City, Calif.) ABI PRISM® BigDye™ Primer or BigDye™ Terminator Cycle Sequencing Kits.

Restriction fragment length polymorphism (RFLP) uses a change in a single nucleotide which modifies a recognition site for a restriction enzyme resulting in the creation or destruction of an RFLP. RFLP can be used on a genomic DNA using a labeled probe (i.e., Southern Blot RFLP) or on a PCR product (i.e., PCR-RFLP).

For example, RFLP can be used to detect the cystic fibrosis—causing mutation, ΔF508 [deletion of a CTT at nucleotide 1653-5, GenBank Accession No. M28668, SEQ ID NO:3; Kerem B, et al., Science. 1989, 245: 1073-80] in a genomic DNA derived from the isolated trophoblast cell of the present invention. Briefly, genomic DNA is amplified using the forward [5′-GCACCATTAAAGAAAATATGAT (SEQ ID NO:4)] and the reverse [5′-CTCTTCTAGTTGGCATGCT (SEQ ID NO:5)] PCR primers, and the resultant 86 or 83 bp PCR products of the wild-type or AF508 allele, respectively are subjected to digestion using the DpnI restriction enzyme which is capable of differentially digesting the wild-type PCR product (resulting in a 67 and 19 bp fragments) but not the CTT-deleted allele (resulting in a 83 bp fragment).

Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

Allele specific oligonucleotide (ASO) is based on an allele-specific oligonucleotide (ASO) which is designed to hybridize in proximity to the substituted nucleotide, such that a primer extension or ligation event can be used as the indicator of a match or a mis-match. Hybridization with radioactively labeled allelic specific oligonucleotides (ASO) also has been applied to the detection of specific SNPs (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles.

It will be appreciated that ASO can be applied on a PCR product generated from genomic DNA. For example, to detect the A455E mutation (C1496→A in SEQ ID NO:3) which causes cystic fibrosis, trophoblast genomic DNA is amplified using the 5′-TAATGGATCATGGGCCATGT (SEQ ID NO:6) and the 5′-ACAGTGTTGAATGTGGTGCA (SEQ ID NO:7) PCR primers, and the resultant PCR product is subjected to an ASO hybridization using the following oligonucleotide probe: 5′-GTTGTTGGAGGTTGCT (SEQ ID NO:8) which is capable of hybridizing to the thymidine nucleotide at position 1496 of SEQ ID NO:3. As a control for the hybridization, the 5′-GTTGTTGGCGGTTGCT (SEQ ID NO:9) oligonucleotide probe is applied to detect the presence of the wild-type allele essentially as described in Kerem B, et al., 1990, Proc. Natl. Acad. Sci. USA, 87:8447-8451).

Allele-specific PCR is based on the detection of the presence of a single nucleic acid substitution using differential extension of a mutant and/or wild-type—specific primer on one hand, and a common primer on the other hand. For example, the detection of the cystic fibrosis Q493X mutation (C1609→T in SEQ ID NO:3) is performed by amplifying genomic DNA (derived from the trophoblast cell of the present invention) using the following three primers: the common primer (i.e., will amplify in any case): 5′-GCAGAGTACCTGAAACAGGA (SEQ ID NO:10); the wild-type primer (i.e., will amplify only the cytosine-containing wild-type allele): 5′-GGCATAATCCAGGAAAACTG (SEQ ID NO:11); and the mutant primer (i.e., will amplify only the thymidine-containing mutant allele): 5′-GGCATAATCCAGGAAAACTA (SEQ ID NO:12), essentially as described in Kerem, 1990 (Supra).

Methylation-specific PCR (MSPCR) is used to detect specific changes in DNA methylation which are associated with imprinting disorders such Angelman or Prader-Willi syndromes. Briefly, the DNA is treated with sodium bisulfite which converts the unmethylated, but not the methylated, cytosine residues to uracil. Following sodium bisulfite treatment the DNA is subjected to a PCR reaction using primers which can anneal to either the uracil nucleotide-containing allele or the cytosine nucleotide-containing allele as described in Buller A., et al., 2000, Mol. Diagn. 5: 239-43.

Pyrosequencing™ analysis (Pyrosequencing, Inc. Westborough, Mass., USA) is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated.

Acycloprime™ analysis (Perkin Elmer, Boston, Mass., USA) is based on fluorescent polarization (FP) detection. Following PCR amplification of the sequence containing the substituted nucleic acid (causing the DNA abnormality in the fetus), excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the substituted nucleic acid. The terminator(s) added are identified by their increased FP and represent the allele(s) present in the original DNA sample. The Acycloprime process uses AcycloPol™, a novel mutant thermostable polymerase from the Archeon family, and a pair of AcycloTerminators™ labeled with R110 and TAMRA, representing the possible alleles for the substituted nucleic acid. AcycloTerminator™ non-nucleotide analogs are biologically active with a variety of DNA polymerases. Similarly to 2′, 3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutants have for derivatized 2′,3′-dideoxynucleotide terminators.

Reverse dot blot uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized colorimetrically after hybridization through the use of streptavidin horseradish peroxidase incubation followed by development using tetramethylbenzidine and hydrogen peroxide, or via chemilumineseence after incubation with avidin alkaline phosphatase conjugate and a luminous substrate susceptible to enzyme activation, such as CSPD, followed by exposure to x-ray film.

Multiplex ligation-dependent probe amplification (MLPA), which is described in Schouten, J. P., et al., 2002 (Nucleic Acids Res. 30: e57), is based on the amplification of 2 probes which are designed to hybridize to adjacent target nucleic acid sequences. Each of the probes includes a gene-specific sequence and common sequence. The common sequences are recognized by the PCR primers. The method is designed to detect single mismatches (which prevent ligation of probes) and the copy number of specific genes. MLPA was shown to detect deletions and/or duplications of exons in the human BRCA1, MSH2 and MLH1 genes, trisomies such as Down's syndrome and characterize chromosomal aberrations.

MS-MLPA (methylation specific MLPA), which is described in Nygren A. O. H., et al., 2005, Nucleic Acid Res. 33: e128, is based on the MLPA analysis but it designed to detect the methylation status of CpG islands (using methylation sensitive enzymes) as well as the copy number of a gene.

It will be appreciated that advances in the field of SNP detection have provided additional accurate, easy, and inexpensive large-scale genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci USA. 88: 7276-80), as well as various DNA “chip” technologies such as the GeneChip microarrays (e.g., Affymetrix SNP chips) which are disclosed in U.S. Pat. Appl. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S, and Higuchi, R. Single-tube genotyping without oligonucleotide probes. Genome Res. 9:72-78 (1999)], FRET primers (Solinas A et al., 2001. Nucleic Acids Res. 29: E96), AlphaScreen [Beaudet L, et al., Genome Res. 2001, 11(4): 600-8], SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut IG. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) and MassArray (Leushner J, Chiu N H, 2000. Mol. Diagn. 5: 341-80).

As is mentioned before, diagnosing according to the method of this aspect of the present invention also encompasses determining the paternity of a fetus. Current methods of testing a prenatal paternity involve obtaining DNA samples from CVS and/or amniocentesis cell samples and subjecting them to PCR-based or RFLP analyses (Strom C M, et al., Am J Obstet. Gynecol. 1996, 174: 1849-53; Yamada Y, et al., 2001. J Forensic Odontostomatol. 19: 1-4).

As used herein, the phrase “paternity” refers to the likelihood that a potential father of a specific fetus is the biological father of that fetus.

Paternity testing (i.e., identification of the paternity of a fetus) according to this aspect of the present invention is achieved by subjecting cell-free fetal nucleus to a genetic analysis capable of detecting polymorphic markers of the fetus, and comparing the fetal polymorphic markers to a set of polymorphic markers obtained from a potential father.

As used herein, the phrase “polymorphic markers” refers to any nucleic acid change (e.g., substitution, deletion, insertion, inversion), variable number of tandem repeats (VNTR), short tandem repeats (STR), minisatellite variant repeats (MVR) and the like.

The polymorphic markers of the present invention can be determined using a variety of methods known in the arts, such as RFLP, PCR, PCR-RFLP and any of the SNP detection methods which are described hereinabove. For example, polymorphic markers used in paternity testing include the minisatellite variant repeats (MVR) at the MS32 (D1S8) or MS31A (D7S21) loci (Tamaki, K et al., 2000, Forensic Sci. Int. 113: 55-62)], the short tandem repeats (STR) at the D1S80 loci (Ceacareanu AC, Ceacareanu B, 1999, Roum. Arch. Microbiol. Immunol. 58: 281-8], the DXYS156 loci (Cali F, et al., 2002, Int. J. Legal Med. 116: 133-8), the “myo” and PYNH24 RFLP-probes [Strom C M, et al., (Supra) and Yamada Y, et al., (Supra)] and/or oligotyping of variable regions such as the HLA-II (Arroyo E, et al., 1994, J. Forensic Sci. 39: 566-72).

It will be appreciated that in order to subject the cell-free fetal nucleus of the present invention to any of the genetic analysis methods described hereinabove, the cell-free fetal nucleus is preferably isolated prior to being subjected to the genetic analysis.

Thus, according to preferred embodiments of this aspect of the present invention, the method further comprising isolating the cell-free fetal nucleus prior to employing an in situ chromosomal and/or DNA analysis and/or genetic analysis.

As used herein, the term “isolating” refers to a physical isolation of a cell-free fetal nucleus from a heterogeneous population of cells or cell-free nuclei. Cell-free fetal nuclei can be isolated from a sample containing maternal cells or nuclei (e.g., maternal blood, transcervical specimens) using a variety of antigen-based methods such as a fluorescence activated cell sorter or immuno-coated beads with a magnetic or electric field (Jamur M C., et al., 2001, J. Histochem. Cytochem. 49: 219-28), all of which utilize antibodies specific to fetal nucleus markers. Alternatively, cell-free fetal nucleus can be isolated in situ (i.e., from a microscopic slide containing such cell-free nuclei) using, for example, laser-capture microdissection.

Laser-capture microdissection of cell-free fetal nucleus is used to selectively isolate a specific cell-free nucleus from a heterogeneous population of cells or cell-free nuclei contained on a slide. Methods of using laser-capture microdissection are known in the art (see for example, U.S. Pat. Appl. No. 20030227611 to Fein, Howard et al., Micke P, et al., 2004. J. Pathol., 202: 130-8; Evans E A, et al., 2003. Reprod. Biol. Endocrinol. 1: 54; Bauer M, et al. 2002. Paternity testing after pregnancy termination using laser microdissection of chorionic villi. Int. J. Legal Med. 116: 39-42; Fend, F. and Raffeld, M. 2000, J. Clin. Pathol. 53: 666-72).

For example, a sample containing a genetic material of a fetus (e.g., transcervical specimen or a blood sample) is contacted with a selectively activated surface [e.g., a thermoplastic membrane such as a polyethylene membrane (PEN slides; C. Zeiss, Thornwood, N.Y.)] capable of adhering to a specific cell-free nucleus upon laser activation. The sample containing the cell-free nuclei is subjected to a differential staining such as an immunological staining (using for example, an MMP9 or Fos-b antibody), followed by a microscopical evaluation (e.g., using an image analysis apparatus) in order to identify the differentially stained cell-free fetal nucleus.

Once identified, a laser beam routed through an optic fiber [e.g., using the PALM Microbeam system (PALM Microlaser Technologies AG, Bernreid, Germany)] activates the surface which adheres to the selected cell-free fetal nucleus. The laser beam uses the ultraviolet (UV, e.g., 337 nm), the far-UV (200-315 nm) and the near-UV (315-400 nm) ray regions which are suitable for the further isolation of DNA, RNA or proteins from the microdissected nucleus. Following dissection (i.e., the cutting off of the cell-free nucleus), the laser beam blows off the cut nucleus into a recovery cap of a microtube, essentially as illustrated in Tachikawa T and Irie T, 2004, Med. Electron Microsc., 37: 82-88. For a genetic analysis, the DNA of the isolated cell-free fetal nucleus can be extracted using e.g., the alkaline lysis or the proteinase K protocols which are described in Rook M, et al., 2004, Am. J. of Pathology, 164: 23-33.

Altogether, the teachings of the present invention can be used to detect chromosomal and/or DNA abnormalities in a fetus, fetal paternity and/or fetal gender by subjecting cell-free fetal nuclei obtained from transcervical or maternal blood specimens to an immunological or RNA-ISH staining method capable of detecting a fetal nucleus-specific marker. Once identified, the cell-free fetal nucleus is subjected to an in situ chromosomal (e.g., FISH, MCB) and/or DNA (e.g., PRINS, Q-FISH) analysis or to nucleus isolation followed by a genetic analysis method such as CGH or any PCR-based detection method.

Briefly, in order to determine chromosomal aberrations and the presence of a cystic fibrosis (CF)— causing mutation in a fetus, cell-free fetal nucleus—containing sample (e.g., transcervical specimen) is subjected to an RNA-ISH staining using an RNA oligonucleotide (e.g., 5′-biotinylated 2′-O-methyl-RNA) designed to hybridize with the H19 RNA transcript (e.g., the 5′-CGUAAUGGAAUGCUUGAAGGCUGC UCCGUGAUGUCGGUCGGAGCUUCCAG-3′ (SEQ ID NO:13). Following hybridization, the cell-free nuclei are viewed under a microscope and the H19-positively stained cell-free nuclei are preferably counterstained and subjected laser capture micro-dissection and isolation. To detect the presence of the CF—causing mutation, the DNA is extracted from the isolated cell-free fetal nucleus using methods known in the arts and is subjected to a PCR-RFLP analysis as described hereinabove. To detect chromosomal aberrations (such as trisomies, duplications, deletions) the DNA extracted from the cell-free fetal nucleus is labeled using e.g., the Spectrum Green-dUTP and is mixed in a 1:1 ratio with a reference DNA (obtained from a normal individual, i.e., 46, XX or 46, XY) which is labeled using the Spectrum Red-dUTP and the mixture of probes is applied on either metaphase chromosomes derived from a normal individual or on a CGH-array, as described hereinabove.

Alternatively, in order to determine chromosomal and DNA abnormalities in a fetus, the RNA-ISH-positively stained fetal nuclei are viewed under a microscope and the locations of the fetal nuclei in the slide are marked and stored. The slides are further subjected to FISH analysis (which detects chromosomal abnormalities), followed by laser micro-dissection and isolation of fetal DNA which can be further subjected to CGH on either metaphase chromosome derived from a normal individual (i.e., 46, XX or 46, XY) or on a CGH-array. Alternatively, for the detection of a single gene disorder or an imprinting disorder, following FISH analysis the DNA of the isolated cell-free fetal nucleus is subjected to any of the PCR-based genetic analysis methods (e.g., ASO, PCR-RFLP, MS-PCR, MLPA and the like).

Still alternatively, prenatal diagnosis of a fetus can be achieved by subjecting the cell-free fetal nuclei of the present invention to an immunological staining using the MMP9 and/or Fos-b antibodies followed by in situ chromosomal and/or DNA analysis (e.g., using PRINS and FISH, MCB or Q-FISH). Additional and/or alternatively, the immunologically stained cell-free fetal nuclei are isolated using laser microdissection and the DNA of the isolated fetal nucleus is subjected to either a CGH analysis (using CGH on metaphase chromosomes or a CGH-array) or to any of the SNP detection methods which are described hereinabove. A non-limiting example of such a sequential genetic analysis is described in Langer et al., 2005, Laboratory Investigation, 85: 582-592; which is fully incorporated herein by reference. Thus, FISH analysis of interphase chromosomes was followed by laser capture microdissection of cells of interest, following which the whole genome of the isolated single cells was further subjected to an unbiased PCR amplification and CGH analysis

To determine the paternity of a fetus, cell-free fetal nuclei are obtained from a pregnant mother and identified as described hereinabove (by ISH-RNA or immunostaining). Once identified, the cell-free fetal nucleus is isolated using laser capture microdissection and the DNA of the isolated fetal nucleus is subjected to a genetic analysis of polymorphic markers such as the D1S80 (MCT118) marker, using the forward: 5′-GAAACTGGCCTCCAAACACTGCCCGCCG (SEQ ID NO:14) or the reverse: 5′-GTCTTGTTGGAGATGCACGTGCCCCTTGC (SEQ ID NO:15) PCR primers, and/or the MS32 and/or the MS31A loci [as described in Tamaki, 2000 (Supra)]. The polymorphic markers of the fetal DNA (i.e., the DNA isolated from the cell-free fetal nucleus of the present invention) are compared to the set of polymorphic markers obtained from the potential father (and preferably also from the mother) and the likelihood of the potential father to be the biological father is calculated using methods known in the art.

Similar analysis can be performed using ethnic-related polymorphic markers (e.g., SNPs in which one allele is exclusively present in a certain ethnic population), which can be used to relate a specific fetus to a potential father of a specific ethnic group (e.g., African Americans) and not to a second potential father of an entirely different ethnic group (e.g., from Iceland).

The agents of the present invention which are described hereinabove for detecting the fetal-nucleus specific marker may be included in a diagnostic kit/article of manufacture preferably along with appropriate instructions for use and labels indicating FDA approval for use in analyzing the genetic material of the fetus.

Such a kit can include, for example, at least one container including a diagnostic reagent for identifying the at least one fetal-nucleus specific marker (e.g., an antibody or a polynucleotide probe) and an imaging reagent packed in another container (e.g., enzymes, secondary antibodies, buffers, chromogenic substrates, fluorogenic material). Preferably, the kit may further include a second reagent for a molecular analysis of the genetic material of the fetus. Such a reagent can be a FISH probe, a PCR primer for genetic analysis, a PNA probe for Q-FISH and the like. The kit may also include appropriate buffers and preservatives for improving the shelf-life of the kit.

The term “antibody” as used in this invention includes intact molecules as well as functional fragments thereof, such as Fab, F(ab′)2, Fv or single domain molecules such as VH and VL capable of specifically binding to an epitope of an antigen (e.g., the fetal-nucleus specific protein). These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference); Goldenberg, U.S. Pat. Nos. 4,036,945 and 4,331,647, and references contained therein, which patents are hereby incorporated by reference in their entirety; Porter, R. R. [Biochem. J. 73: 119-126 (1959)]; Inbar et al. [Proc. Nat'l Acad. Sci. USA 69:2659-62 (19720]; Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety; Larrick and Fry [Methods, 2: 106-10 (1991)].

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Prenatal Diagnosis by Molecular Analysis of Cell-Free Fetal Nuclei Present in Transcervical Samples

Material and Experimental Methods

Study subjects—Pregnant women between 5^{th} and 15^{th} week of gestation, which were either scheduled to undergo a pregnancy termination or were invited for a routine check-up of an ongoing pregnancy, were enrolled in the study after giving their informed consent. It will be appreciated that transcervical cell-free nuclei can be also obtained from transcervical specimens obtained at earlier stages of pregnancy such as from 4 and even 3 weeks of gestation.

Sampling of transcervical specimens—A Pap smear cytobrush (MedScand-AB, Malmö, Sweden/bioteque America, Inc.) was inserted through the external os to a maximum depth of 2 cm (the brush's length), and removed while rotating it a full turn (i.e., 360°). In order to remove the transcervical cells caught on the brush, the brush was shaken into a test tube containing 2-3 ml of the RPMI-1640 medium (Beth Haemek, Israel) in the presence of 1% Penicillin Streptomycin antibiotic. Cytospin slides (8 slides from each transcervical specimen—2 circles on each slide) were then prepared by dripping 1-3 drops of the RPMI-1640 medium containing the transcervical cell-free nuclei into the Cytofunnel Chamber Cytocentrifuge (Wescor, Canada). The conditions used for cytocentrifugation were dependent on the murkiness of the transcervical specimen. The cytospin slides were kept in 95% alcohol.

Immunohistochemical (IHC) staining of transcervical specimens—Cytospin slides containing the transcervical cells and cell-free nuclei were washed in 70% alcohol solution and dipped for 5 minutes in distilled water. All washes in PBS, including blocking reagent were performed while gently shaking the slides. The slides were then transferred into a moist chamber, washed with phosphate buffered-saline (PBS). To visualize the position of the cells or the cell-free-nuclei on the microscopic slides, the borders of the transcervical specimens were marked using a Pap Pen (Zymed Laboratories Inc., San Francisco, Calif., USA). 100 μl of 3% hydrogen peroxide (Merck, Germany) were added to each slide for a 5-minute incubation at room temperature following which the slides were washed in PBS. To avoid non-specific binding of the antibody, two drops of a blocking reagent (Zymed HISTOSTAIN®-PLUS Kit, Cat No. 858943) were added to each slide for a 5-minute incubation in a moist chamber. To identify the fetal trophoblast cells or cell-free nuclei in the transcervical specimen 100 μl of individual antibodies or mixed antibodies were applied to each slide. The antibody's mix included: an HLA-G antibody (Serotec, USA, Cat. No MCA2043 or Exbio, Czech Republic Cat. No. 11291M001, 1 mg/ml) part of the non-classical class I major histocompatibility complex (MHC) antigen specific to extravillous trophoblast cells (Loke, Y. W. et al., 1997. Tissue Antigens 50: 135-146) diluted 1:150 in antibody diluent solution (Zymed), Mouse anti human trophoblast protein (NDOG1) (Serotec immunological excellence, USA, Cat. No. MCA277), antigen specific to syncytium trophoblast, diluted 1:50 in antibody diluent solution (Zymed), or Ab 340.0.9 mg/ml (Dr Lindy Durrant, University of Nottingham, UK) antigen specific to syncytium trophoblast and cytotrophoblast, diluted 1:277 in antibody diluent solution (Zymed) and CD146 (Alexis, Switzerland, Cat No. ALX-805-031) antigen specific to extravillous trophoblast, diluted 1:100 in antibody diluent solution (Zymed). Other antibodies which are specific to the nucleus of fetal cells (e.g., trophoblast) include the MMP9 (2C3; monoclonal IgG, SC-21733, Santa Cruz), Fos-b (C-11; monoclonal IgM, SC-8013, Santa Cruz), and C-Jun (Novus). The slides were incubated with the antibody in a moist chamber for 60 minutes, following which they were washed with PBS. To detect the bound primary antibody, two drops of a secondary biotinylated goat anti-mouse IgG antibody (Zymed HISTOSTAIN®-PLUS Kit, Cat No. 858943) were added to each slide for a 15-minute incubation in a moist chamber. The secondary antibody was washed with PBS. To reveal the biotinylated secondary antibody, two drops of an horseradish peroxidase (HRP)-streptavidin conjugate (Zymed HISTOSTAIN®-PLUS Kit, Cat No. 858943) were added for a 15-minute incubation in a moist chamber, and washed in PBS. Finally, to detect the HRP-conjugated streptavidin, three drops of an aminoethylcarbazole (AEC Single Solution Chromogen/Substrate, Zymed) HRP substrate was added for a 10-minute incubation in a moist chamber, followed by three washed with PBS.

Nuclei counterstaining—Counterstaining was performed by adding for 25 seconds two drops of 2% Hematoxylin solution [Sigma-Aldrich Corp., St Louis, Mo., USA, Cat. No. GHS-2-32 (Mixture of 730 ml Distilled water, 250 ml Ethylene glycol, 2 grams hematoxylin, 0.2 grams Sodium iodate and 17.6 grams Aluminum sulfate, Merck, Germany)] following which the slides were washed under tap water and covered with a coverslip.

Microscopic analysis and morphological evaluation of stained cell-free nuclei—Immuno-stained and counterstained slides containing the transcervical cells and cell-free nuclei were scanned using a light microscope (BX61, Olympus, Japan) and the BioView Duet™ image analysis apparatus (Bio View Ltd. Rehovot, Israel). The location of cell-free nuclei was marked using the coordination numbers in the microscope.

Removal of antibody residual staining and counterstaining following immunohistochemistry and prior to FISH analysis using ammonium hydroxide—Antibody residual staining was removed using ammonium hydroxide. Briefly, following immunohistochemistry, slides were dipped for 5-10 minutes in double-distilled water until the coverslips were gently removed from slides. Stained slides were then incubated for 45 minutes in solution of 2% ammonium hydroxide (diluted in 70% alcohol), washed in double-distilled water and dehydrated in increasing ethanol concentrations of 70% and 100% ethanol, 2 minutes each.

Following dehydration, the specimens were fixed for 45 minutes in an ice-cold methanol-acetic acid (at a 3:1 ratio, respectively) fixer solution (Merck). Following fixation, the specimens were dried at room temperature.

Pre-Treatment of Immunohistochemical Stained Slides Prior to FISH Analysis

Fixed slides (following ammonium hydroxide treatment and dehydration) were dipped for 15 minutes in a warm solution (at 37° C.) of 300 mM NaCl, 30 mM NaCltrate (2×SSC) at pH 7.0-7.5. Following incubation, the excess of the 2×SSC solution was drained off and the slides were fixed for 15 minutes at room temperature in a solution of 0.9% of formaldehyde in PBS. Slides were then washed for 5 minutes in PBS and the cell-free nuclei were digested for 14 minutes at 37° C. in a solution of 0.15% of Pepsin (Sigma) in 0.01 N HCl. Following Pepsin digestion slides were washed for 5 minutes in PBS and were allowed to dry. To ensure a complete dehydration, the slides were dipped in a series of 70%, 85% and 100% ethanol (1 minute each), and dried (using Hot plate) at 45-50° C.

FISH probes—FISH analysis was carried out using AneuVysion probe (Vysis, Abbott, Germany, Cat No. 35-161075): CEP probes for chromosome 18 (Aqua), X (green), Y (orange) and repeated FISH (in Ongoing pregnancy) with LSI probes for 13 green and 21 orange.

For FISH analysis, 3 μl of the CEP probes of chromosome 18 (Aqua), X (green), Y (orange) were applied to the target areas and covered with a round 13 mm coverslip. In situ hybridization was carried out in the HYBrite apparatus (Abbott Cat. No. 2J11-04) by setting the melting temperature to 71° C. and the melting time for 4 minutes. The hybridization was carried out for 48-60 hours at 37° C.

Following hybridization, slides were washed for 2 minutes at 70° C. in a solution of 0.3% NP-40 (Abbott) in 0.4×SSC (60 mM NaCl and 6 mM NaCitrate). Slides were then immersed for 1 minute in a solution of 0.1% NP-40 in 2×SSC at room temperature, following which the slides were allowed to dry in the darkness. Counterstaining was performed using 8 μl of a DAPI II counterstain (Abbott), following which the slides were covered using a coverslip.

Experimental Results

Identification of multiple cell-free nuclei in transcervical specimens—

FIGS. 1-4 present transcervical samples obtained from pregnant women at the 6^{th} (FIGS. 1, 2) or 8^{th} (FIGS. 3, 4) week of gestation. The samples were subjected to IHC using the HLA-G antibody (mAb 7759, Abcam) and Hematoxylin counterstaining, followed by FISH analysis using the CEP X and CEP Y or SatIII Y (Abbott, Cat. 5J10-51) probes and DAPI counterstaining as described in material and methods above.

Only transcervical specimens obtained from women later identified as carrying male fetuses were analyzed. The selection of specimen containing male fetal cells was done in order to provide a non biased identifier of fetal material (cells having XY chromosomes must be of a fetal origin). There is no limitation in performing similar analyses with female fetuses.

Analysis of the samples revealed that many fetal cells (identified by their male XY genotype) have lost their cytoplasm and are present as cell-free nuclei. The ratio between embryonic whole cells (nuclei+cytoplasm, identified using IHC) and embryonic nuclei was found to be at least about 1:10, i.e. 3 whole cells versus at least 28 nuclei in average per PAP sample, as measured using the CEP X and Sat III Y probes.

Thus, the present inventors have uncovered that cell-free nuclei are present in the transcervical specimens and that they are readily amenable to FISH analysis.

These findings imply the use of cell-free fetal nuclei for analyzing the genetic material of a fetus and hence their use for non-invasive prenatal diagnosis.

### Example 2

### Ash2-Immunofluorescence as a Tool for the Identification of Fetal Cell Free Nuclei

Immunofluorescence Staining—PAP samples were collected from pregnant women (5-12 weeks of gestation) and kept in 2% PFA till they arrive at the lab (about 2 h). Samples were washed twice in 1×PBS 2000 g 5 min. Pellets were resuspended in 2 ml 1×PBS and aliquots of 100 μl were fixed on a slide using the Cytospin centrifuge 1500 RPM, 5 min. Slides were immersed in blocking solution (1×PBS/3% BSA/0.5% Triton X-100) for 45 min. RT, followed by incubation with first antibody (anti Ash2 rabbit polyclonal antibody, Novus NB600-253, 1 mg/ml in Blocking solution) for 1 hr RT. Next, the slides were washed with 1×PBS twice for 5 min. and subjected to incubation with Cy2 conjugated secondary antibody (1.5 mg/ml, Jackson Laboratories) for 1 h RT. Cells were washed twice with 1×PBS for 5 min. and counterstained with DAPI (1 ug/ml in 1×PBS, Sigma) for 30 min. RT. Slides were mounted and subjected to microscopic analysis followed by FISH analysis using the CEP X spectrum green and CEP Y spectrum orange probes (Abbot, cat. 5J10-51) for identification of male embryonic cells.

Experimental results—One way of differentially visualizing fetal and maternal cell-free nuclei is by staining with an antibody directed against the fetal specific Ash 2 nuclear membrane protein (Cross et al. 2003 Placenta, 24 (2-3): 123-30). FIG. 5 shows Ash 2 protein staining of the nucleus membrane followed by FISH analysis using the CEP X and CEP Y (Abbott, Cat. 5J10-51) probes and DAPI counterstaining as described in material and methods above.

### Example 3

H19 RNA-ISH as a Tool for the Identification of Fetal Cell Free Nuclei

The H19 gene is known to be expressed in fetal-placental cells (Goshen R., et al., 1993, Mol. Reprod. Dev. 34: 374-9; Poirier F., et al., 1991, Development, 113: 1105-14; Xiao Yang, et al., 1998, Proc. Natl. Acad. Sci. U.S.A. 95: 3667-3672) but not in epithelial cells of normal endometrium (Ariel I., et al., Am J Med Genet. 2000, 91:46-50; Tanos V, et al., 2004, Int. J. Gynecol. Cancer, 14: 521-5). To-date, no reports of H19 expression in the maternal decidua were found (http://www.genecards.org/);

Transcervical specimens are obtained from pregnant women at the 7^{th} week of gestation (as described in Example 1, hereinabove) and are subjected to a non-radioactive RNA-ISH using a Digoxigenin-labeled H19 RNA probe essentially as described in Ariel I., et al., 1998, J. Clin. Pathol. Mol. Pathol. 51:21-25 which is fully incorporated herein by reference.

Further molecular analysis on the identified cell-free fetal nuclei can be done using, for example, FISH, MCB, Q-FISH and CGH analyses (as described hereinabove) to thereby determine fetal gender and/or chromosomal abnormalities. In addition, the identified cell-free fetal nucleus (e.g., the H19-positive nucleus) can be isolated using e.g., laser capture microdissection and its DNA can be further subject to any DNA-based molecular analysis which can be further used in the diagnosis of various genetic diseases such as single gene disorders (e.g., cystic fibrosis), predisposition to cancers (e.g., BRCA1) and determination of fetal paternity.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of identifying a fetal nucleus, comprising:
(a) detecting a cell-free nucleus in a sample; and
(b) detecting in a cell-free nucleus at least one fetal-nucleus specific marker;
thereby identifying the fetal nucleus.

2. The method of claim 1, wherein said sample is a trophoblast-containing sample obtained from a pregnant woman.

3. The method of claim 2, wherein said trophoblast-containing sample is obtained from a cervix and/or a uterus of said pregnant woman.

4. The method of claim 2, wherein said trophoblast-containing sample is obtained using a method selected from the group consisting of aspiration, cytobrush, cotton wool swab, endocervical lavage and intrauterine lavage.

5. The method of claim 1, wherein said sample is a blood sample obtained from a pregnant woman.

6. The method of claim 1, wherein said at least one fetal-nucleus specific marker is a molecular marker.

7. The method of claim 6, wherein said molecular marker is selected from the group consisting of a nucleic acid marker and a protein marker.

8. The method of claim 7, wherein said nucleic acid marker is a nuclear RNA molecule.

9. The method of claim 8, wherein said nuclear RNA molecule is an H19 transcript.

10. The method of claim 7, wherein said nucleic acid marker is an epigenetic marker.

11. The method of claim 10, wherein said epigenetic marker is located on H19 and/or IGF2.

12. The method of claim 8, wherein said nuclear RNA molecule is an hnRNA transcript encoding a polypeptide selected from the group consisting of ESX1L, MASH2, Ash2, Stra 13, FosB, Cyclin D1, GCM1 and Caspase-8.

13. The method of claim 7, wherein said protein marker is a trophoblast specific antigen selected from the group consisting of ESX1L, MASH2, Ash2, Stra 13, FosB, Cyclin D1, GCM1 and Caspase-8.

14. The method of claim 7, wherein detection of said nuclear RNA molecule comprises using an RNA in situ hybridization (RNA-ISH) staining.

15. The method of claim 14, wherein said RNA-ISH staining comprises using a probe selected from the group consisting of an RNA molecule, a DNA molecule and a PNA oligonucleotide.

16. The method of claim 15, wherein said RNA molecule is an RNA oligonucleotide and/or an in vitro transcribed RNA.

17. The method of claim 15, wherein said DNA molecule is an oligonucleotide and/or a cDNA molecule.

18. The method of claim 7, wherein detecting said protein marker comprises using an immunological staining.

19. The method of claim 2, wherein said trophoblast-containing sample is obtained from a pregnant woman at 5^{th} to 15^{th} week of gestation.

20. The method of claim 1, where said identifying said at least one cell-free nucleus in said sample is achieved by image analysis.

21. A method of analyzing a genetic material of a fetus, comprising:
(a) detecting a cell-free nucleus in a sample and;
(b) detecting in a cell-free nucleus at least one fetal-nucleus specific marker;
thereby identifying a fetal nucleus; and
(c) molecularly analyzing the genetic material in said fetal nucleus;
thereby analyzing the genetic material of the fetus.

22. The method of claim 21, wherein said sample is a trophoblast-containing sample obtained from a pregnant woman.

23. The method of claim 22, wherein said trophoblast-containing sample is obtained from a cervix and/or a uterus of said pregnant woman.

24. The method of claim 22, wherein said trophoblast-containing sample is obtained using a method selected from the group consisting of aspiration, cytobrush, cotton wool swab, endocervical lavage and intrauterine lavage.

25. The method of claim 21, wherein said sample is a blood sample obtained from a pregnant woman.

26. The method of claim 21, wherein said at least one fetal-nucleus specific marker is a molecular marker.

27. The method of claim 26, wherein said molecular marker is selected from the group consisting of a nucleic acid marker and a protein marker.

28. The method of claim 27, wherein said nucleic acid marker is a nuclear RNA molecule.

29. The method of claim 28, wherein said nuclear RNA molecule is an H19 transcript.

30. The method of claim 27, wherein said nucleic acid marker is an epigenetic marker.

31. The method of claim 30, wherein said epigenetic marker is located on H19 and/or IGF2.

32. The method of claim 28, wherein said nuclear RNA molecule is an hnRNA transcript encoding a polypeptide selected from the group consisting of ESX1L, MASH2, Ash2, Stra 13, FosB, Cyclin D1, GCM1 and Caspase-8.

33. The method of claim 27, wherein said protein marker is a trophoblast specific antigen selected from the group consisting of ESX1L, MASH2, Ash2, Stra 13, FosB, Cyclin D1, GCM1 and Caspase-8.

34. The method of claim 27, wherein detection of said nuclear RNA molecule comprises using an RNA in situ hybridization (RNA-ISH) staining.

35. The method of claim 34, wherein said RNA-ISH staining comprises using a probe selected from the group consisting of an RNA molecule, a DNA molecule and a PNA oligonucleotide.

36. The method of claim 35, wherein said RNA molecule is an RNA oligonucleotide and/or an in vitro transcribed RNA.

37. The method of claim 35, wherein said DNA molecule is an oligonucleotide and/or a cDNA molecule.

38. The method of claim 27, wherein detecting said protein marker comprises using an immunological staining.

39. The method of claim 22, wherein said trophoblast-containing sample is obtained from a pregnant woman at 5^{th} to 15^{th} week of gestation.

40. The method of claim 21, wherein said molecularly analyzing said genetic material comprises using an approach selected from the group consisting of an in situ chromosomal analysis, an in situ DNA analysis and a genetic analysis.

41. The method of claim 40, wherein said in situ chromosomal analysis comprises using fluorescent in situ hybridization (FISH) and/or multicolor-banding (MCB).

42. The method of claim 40, wherein said in situ DNA analysis comprises using primed in situ labeling (PRINS) and/or quantitative FISH (Q-FISH).

43. The method of claim 42, wherein said Q-FISH comprises using a peptide nucleic acid (PNA) oligonucleotide probe.

44. The method of claim 40, wherein said genetic analysis utilizes at least one method selected from the group consisting of comparative genome hybridization (CGH) and identification of at least one nucleic acid substitution.

45. The method of claim 21, further comprising a step of isolating said at least one fetal nucleus prior to step (c).

46. The method of claim 45, wherein said isolating said at least one fetal nucleus is achieved using laser microdissection.

47. The method of claim 44, wherein said identification of at least one nucleic acid substitution is achieved using a method selected from the group consisting of DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, Invader assay, MLPA and MS-MLPA.

48. The method of claim 21, wherein said identifying said at least one cell-free nucleus in said sample is achieved by image analysis.

49. The method of claim 21, wherein analysis of the genetic material of the fetus enables the identification of fetal gender, at least one chromosomal abnormality, at least one DNA abnormality and/or a paternity of the fetus.

50. The method of claim 49, wherein said at least one chromosomal abnormality is selected from the group consisting of aneuploidy, translocation, subtelomeric rearrangement, unbalanced subtelomeric rearrangement, deletion, microdeletion, inversion, duplication, and telomere instability and/or shortening.

51. The method of claim 50, wherein said chromosomal aneuploidy is a complete and/or partial trisomy.

52. The method of claim 51, wherein said trisomy is selected from the group consisting of trisomy 21, trisomy 18, trisomy 13, trisomy 16, XXY, XYY, and XXX.

53. The method of claim 50, wherein said chromosomal aneuploidy is a complete and/or partial monosomy.

54. The method of claim 53, wherein said monosomy is selected from the group consisting of monosomy X, monosomy 21, monosomy 22, monosomy 16 and monosomy 15.

55. The method of claim 49, wherein said at least one DNA abnormality is selected from the group consisting of single nucleotide substitution, micro-deletion, micro-insertion, short deletions, short insertions, multinucleotide changes, DNA methylation and loss of imprint (LOI).

56. A kit for analyzing a genetic material of a fetus, comprising a packaging material packaging a reagent for detecting at least one fetal-nucleus specific marker.

57. The kit of claim 56, wherein the genetic material of the fetus is derived from a cell-free fetal nucleus.

58. The method of claim 56, wherein said at least one fetal-nucleus specific marker is a molecular marker.

59. The kit of claim 58, wherein said molecular marker is selected from the group consisting of a nucleic acid marker and a protein marker.

60. The kit of claim 59, wherein said nucleic acid marker is a nuclear RNA molecule.

61. The kit of claim 60, wherein said nuclear RNA molecule is an H19 transcript.

62. The kit of claim 59, wherein said nucleic acid marker is an epigenetic marker.

63. The kit of claim 62, wherein said epigenetic marker is located on H19 and/or IGF2.

64. The kit of claim 60, wherein said nuclear RNA molecule is an hnRNA transcript encoding a polypeptide selected from the group consisting of ESX1L, MASH2, Ash2, Stra 13, FosB, Cyclin D1, GCM1 and Caspase-8.

65. The kit of claim 59, wherein said protein marker is a trophoblast specific antigen selected from the group consisting of ESX1L, MASH2, Ash2, Stra 13, FosB, Cyclin D1, GCM1 and Caspase-8.

66. The kit of claim 60, wherein detection of said nuclear RNA molecule comprises using an RNA in situ hybridization (RNA-ISH) staining.

67. The kit of claim 66, wherein said RNA-ISH staining comprises using a probe selected from the group consisting of an RNA molecule, a DNA molecule and a PNA oligonucleotide.

68. The kit of claim 67, wherein said RNA molecule is an RNA oligonucleotide and/or an in vitro transcribed RNA.

69. The kit of claim 67, wherein said DNA molecule is an oligonucleotide and/or a cDNA molecule.

70. The kit of claim 59, wherein detection of said protein marker comprises using an immunological staining.

71. The kit of claim 56, further comprising a second reagent suitable for a molecular analysis of the genetic material of the fetus, said molecular analysis is selected from the group consisting of an in situ chromosomal analysis, an in situ DNA analysis and a genetic analysis.

72. The kit of claim 71, wherein said in situ chromosomal analysis comprises using fluorescent in situ hybridization (FISH) and/or multicolor-banding (MCB).

73. The kit of claim 71, wherein said in situ DNA analysis comprises using primed in situ labeling (PRINS) and/or quantitative FISH (Q-FISH).

74. The kit of claim 73, wherein said Q-FISH comprises using a peptide nucleic acid (PNA) oligonucleotide probe.

75. The kit of claim 71, wherein said genetic analysis utilizes at least one method selected from the group consisting of comparative genome hybridization (CGH) and identification of at least one nucleic acid substitution.

76. The kit of claim 75, wherein said identification of at least one nucleic acid substitution is achieved using a method selected from the group consisting of DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, Invader assay, MLPA and MS-MLPA.
